(19) United States
(12) Patent Application Publication
(10) Pub. No.: US 2024/0190990 A1
(43) Pub. Date: Jun. 13, 2024

(54) ANTI-CEA ANTIBODIES AND METHODS OF USE

(71) Applicant: BeiGene Switzerland GmbH, Basel (CH)

(72) Inventors: Liang QU, Beijing (CN); Zhuo LI, Shanghai (CN); Liu XUE, Beijing (CN); Qi LIU, Beijing (CN); Lin ZHU, Beijing (CN); Penghao WANG, Beijing (CN); Hanzi SUN, Beijing (CN); Xiaoyan TANG, Beijing (CN)

(73) Assignee: BeiGene Switzerland GmbH, Basel (CH)

(21) Appl. No.: 18/513,255

(22) Filed: Nov. 17, 2023

Related U.S. Application Data
(63) Continuation of application No. PCT/CN2022/093566, filed on May 18, 2022.

(30) Foreign Application Priority Data
May 21, 2021 (WO) PCT/CN2021/095113
Apr. 21, 2022 (WO) PCT/CN2022/088175

(51) Int. Cl. C07K 16/30; A61K 39/00; A61K 45/06; A61K 47/64; A61P 35/00

(52) U.S. Cl. CPC C07K 16/3007; A61K 45/06; A61K 47/6415; A61P 35/00; A61K 2039/505; C07K 2317/52; C07K 2317/732; C07K 2317/734

(57) ABSTRACT

The present disclosure provides for antibodies and antigen-binding fragments thereof that bind to human CEA, a pharmaceutical composition comprising said antibody, and use of the anti-CEA antibody or the composition for treating a disease, such as cancer.

### CROSS-REFERENCE TO RELATED APPLICATIONS

This application is a continuation of International Patent Application No. PCT/CN2022/093566, filed May 18, 2022, which claims priority from International Patent Application No. PCT/CN2021/095113, filed May 21, 2021, and International Patent Application No. PCT/CN2022/088175, filed Apr. 21, 2022. The contents of these applications are incorporated herein by reference in their entirety.

### SEQUENCE LISTING

The instant application contains a Sequence Listing which has been submitted electronically in XML format and is hereby incorporated by reference in its entirety. Said XML file was created on Jan. 2, 2024, is named 138881_0251_Sequence_Listing.xml, and is 142 bytes in size.

### FIELD OF THE DISCLOSURE

Disclosed herein are antibodies or antigen-binding fragments thereof that bind to human CEA as well as methods of use for the treatment of cancer.

### BACKGROUND

Carcinoembryonic antigen (CEA, also known as CEACAM5 or CD66e) is a glycoprotein with a molecular weight of about 70-100 kDa depending on the amount of glycosylation present. The presence of CEA associated as cancer-specific antigen in human adenocarcinoma was first reported by Gold et al., J. Exp. Med., 121, 439 (1965). CEA is normally expressed in a variety of glandular epithelial tissues (such as the gastrointestinal, respiratory, and urogenital tracts) where it appears to be localized to the apical surface of the cells (Hammarstrom, S. Semin. Cancer Biol. 9, 67-81 (1999)). For example, it is found in the columnar epithelial and goblet cells of the colon (Fraengsmyr et al., Tumor Biol. 20:277-292(1999)). In tumors generated from these tissue types, CEA expression increases from the apical membrane to the cell surface and once removed from the cell surface, enters into the bloodstream (Hammarstrom, S. Semin. Cancer Biol. 9, 67-81; (1999) see also Fraengsmyr et al., Tumor Biol. 20:277-292(1999)). CEA overexpression was observed in many types of cancers, including colorectal cancer, pancreatic cancer, lung cancer, gastric cancer, hepatocellular carcinoma, breast cancer, and thyroid cancer. Therefore, CEA has been useful as a diagnostic tumor marker to determine the elevated levels of CEA in the blood of cancer patients in the prognosis and management of cancer (Chevinsky, A. H. (1991) Semin. Surg. Oncol. 7, 162-166; Shively, J. E. et al., (1985) Crit. Rev. Oncol. Hematol. 2, 355-399).

CEA has been considered as a useful tumor-associated antigen for targeted therapy (Kuroki M, et al., (2002) Anticancer Res 22:4255-64). One approach was the generation of retrovirus constructs that displayed an anti-CEA scFv, and would deliver a nitric oxide synthase (iNOS) gene to CEA expressing cancer cells. (Kuroki M. et al., (2000) Anticancer Res. 20(6A):4067-71). Another approach was to attach radioisotopes to anti-CEA antibodies and demonstrate that radiation was directed specifically at the CEA expressing tumor (Wilkinson et al., PNAS USA 98, 10256-60 (2001), Goldenberg et al., Am. J. Gastroenterol., 86: 1392-1403 (1991), Olafsen T. et al., Protein Engineering, Design & Selection, 17, 21-27, (2004), Meyer et al., Clin. Cancer Res. 15:4484-4492 (2009), Sharkey et al., J. Nucl. Med. 46:620-633 (2005)). The radioisotope approach has been extended to anti-CEA antibody drug conjugates (ADC). For example, Shinmi et al., reported on an anti-CEA antibody conjugated to monomethyl auristatin E (MMAE) (Shinmi et al., Cancer Med. 6(4): 798-808 (2017)).

However, one of the issues for anti-CEA antibodies is that of cross-reactivity. CEA is highly homologous to other CEACAM family members, for example, human CEA shows 84% homology with CEACAM6, 77% homology with CECAM8 and 73% identity with CEACAM1. The current disclosure provides for anti-CEA antibodies that are specific for CEA.

### SUMMARY OF THE DISCLOSURE

The present disclosure is directed to anti-CEA antibodies and antigen-binding fragments thereof. The present disclosure encompasses the following embodiments.

An anti-CEA antibody or antigen-binding fragment thereof, comprising an antibody or binding fragment thereof that specifically binds to human CEA at amino acids 596 to 674 of SEQ ID NO:52.

The anti-CEA antibody or antigen-binding fragment, wherein the antibody or antigen-binding fragment does not bind to other CEACAM family members.

The anti-CEA antibody or antigen-binding fragment of claim 1, comprising:
(i) a heavy chain variable region that comprises (a) a HCDR1 (Heavy Chain Complementarity Determining Region 1) of SEQ ID NO: 7, (b) a HCDR2 of SEQ ID NO:8, (c) a HCDR3 of SEQ ID NO:9 and a light chain variable region that comprises: (d) a LCDR1 (Light Chain Complementarity Determining Region 1) of SEQ ID NO: 10, (e) a LCDR2 of SEQ ID NO:11, and (f) a LCDR3 of SEQ ID NO:6;
(ii) a heavy chain variable region that comprises (a) a HCDR1 of SEQ ID NO:24, (b) a HCDR2 of SEQ ID NO:25, (c) a HCDR3 of SEQ ID NO:26; and a light chain variable region that comprises: (d) a LCDR1 of SEQ ID NO:27, (e) a LCDR2 of SEQ ID NO:28, and (f) a LCDR3 of SEQ ID NO: 23; or
(iii) a heavy chain variable region that comprises (a) a HCDR1 of SEQ ID NO:41, (b) a HCDR2 of SEQ ID NO:42, (c) a HCDR3 of SEQ ID NO:43; and a light chain variable region that comprises: (d) a LCDR1 of SEQ ID NO:44, (e) a LCDR2 of SEQ ID NO:45, and (f) a LCDR3 of SEQ ID NO:40.

The anti-CEA antibody or antigen-binding fragment, comprising:
(i) a heavy chain variable region (VH) comprising an amino acid sequence at least 90, 91, 92, 93, 94, 95, 96, 97, 98 or 99% identical to SEQ ID NO:14, and a light chain variable region (VL) comprising an amino acid sequence at least 90, 91, 92, 93, 94, 95, 96, 97, 98 or 99% identical to SEQ ID NO: 15;
(ii) a heavy chain variable region (VH) comprising an amino acid sequence at least 90, 91, 92, 93, 94, 95, 96, 97, 98 or 99% identical to SEQ ID NO: 31, and a light chain variable region (VL) comprising an amino acid sequence at least 90, 91, 92, 93, 94, 95, 96, 97, 98 or 99% identical to SEQ ID NO: 32; or
(iii) a heavy chain variable region (VH) comprising an amino acid sequence at least 90, 91, 92, 93, 94, 95, 96, 97, 98 or 99% identical to SEQ ID NO: 48, and a light chain variable region (VL) comprising an amino acid sequence at least 90, 91, 92, 93, 94, 95, 96, 97, 98 or 99% identical to SEQ ID NO: 49.

The anti-CEA antibody or antigen-binding fragment, wherein one, two, three, four, five, six, seven, eight, nine, or ten amino acids within SEQ ID NO: 14, 15, 31, 32, 48, or 49 have been inserted, deleted or substituted.

The anti-CEA antibody or antigen-binding fragment, comprising:
(i) a heavy chain variable region (VH) that comprises SEQ ID NO:14, and a light chain variable region (VL) that comprises SEQ ID NO: 15;
(ii) a heavy chain variable region (VH) that comprises SEQ ID NO: 31, and a light chain variable region (VL) that comprises SEQ ID NO: 32; or
(iii) a heavy chain variable region (VH) that comprises SEQ ID NO: 48, and a light chain variable region (VL) that comprises SEQ ID NO: 49.

The anti-CEA antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)2 fragment.

The anti-CEA antibody or antigen-binding fragment, wherein the antibody or antigen-binding fragment thereof has antibody dependent cellular cytotoxicity (ADCC) or complement dependent cytotoxicity (CDC).

The anti-CEA antibody or antigen-binding fragment, wherein the antibody or antigen-binding fragment thereof has reduced glycosylation or no glycosylation or is hypofucosylated.

The anti-CEA antibody or antigen-binding fragment, wherein the antibody or antigen-binding fragment thereof comprises increased bisecting GlcNac structures.

The anti-CEA antibody or antigen-binding fragment, wherein the Fc domain is an IgG1.

The anti-CEA antibody or antigen-binding fragment, wherein the antibody is conjugated to a toxin.

A pharmaceutical composition comprising the anti-CEA antibody or antigen-binding fragment thereof, further comprising a pharmaceutically acceptable carrier.

A method of treating cancer comprising administering to a patient in need an effective amount of the antibody or antigen-binding fragment.

The method wherein the cancer is gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The method wherein the antibody or antigen-binding fragment is administered in combination with another therapeutic agent.

The method wherein the therapeutic agent is paclitaxel or a paclitaxel agent, docetaxel, carboplatin, topotecan, cisplatin, irinotecan, doxorubicin, lenalidomide or 5-azacytidine.

The method wherein the therapeutic agent an anti-PD1 or anti-PDL1 antibody.

An isolated nucleic acid that encodes the anti-CEA antibody or antigen-binding fragment.

A vector comprising the nucleic acid.

A host cell comprising the nucleic acid or the vector.

A process for producing an anti-CEA antibody or antigen-binding fragment thereof comprising cultivating the host cell and recovering the antibody or antigen-binding fragment from the culture.

In one embodiment, the anti-CEA antibody or an antigen-binding fragment thereof comprises one or more complementarity determining regions (CDRs) comprising an amino acid sequence selected from a group consisting of SEQ ID NO:7, SEQ ID NO:8, SEQ ID NO:9, SEQ ID NO: 10, SEQ ID NO: 11, SEQ ID NO:6, SEQ ID NO:24, SEQ ID NO:25, SEQ ID NO:26, SEQ ID NO:27, SEQ ID NO:28, SEQ ID NO:23, SEQ ID NO:41, SEQ ID NO:42, SEQ ID NO:43, SEQ ID NO:44, SEQ ID NO:45 or SEQ ID NO:40.

In another embodiment, the antibody or an antigen-binding fragment thereof comprises: (a) a heavy chain variable region comprising one or more complementarity determining regions (HCDRs) comprising an amino acid sequence selected from the group consisting of SEQ ID NO: 7, SEQ ID NO: 8, SEQ ID NO: 9, SEQ ID NO: 24, SEQ ID NO: 25, SEQ ID NO: 26, SEQ ID NO: 41, SEQ ID NO: 42, or SEQ ID NO: 43; and/or (b) a light chain variable region comprising one or more complementarity determining regions (LCDRs) having an amino acid sequence selected from the group consisting of SEQ ID NO: 10, SEQ ID NO: 11, SEQ ID NO: 6, SEQ ID NO: 27, SEQ ID NO: 28, SEQ ID NO: 23, SEQ ID NO: 44 SEQ ID NO: 45 or SEQ ID NO: 40.

In another embodiment, the antibody or an antigen-binding fragment thereof comprises: (a) a heavy chain variable region comprising three complementarity determining regions (HCDRs) which are HCDR1 comprising an amino acid sequence of SEQ ID NO: 7; SEQ ID NO: 24 or SEQ ID NO:41, HCDR2 comprising an amino acid sequence of SEQ ID NO: 8, SEQ ID NO: 25 or SEQ ID NO: 42, and HCDR3 comprising an amino acid sequence of SEQ ID NO: 9, SEQ ID NO: 26 or SEQ ID NO:43 and/or (b) a light chain variable region comprising three complementarity determining regions (LCDRs) which are LCDR1 comprising an amino acid sequence of SEQ ID NO: 10, SEQ ID NO: 27, or SEQ ID NO: 44, LCDR2 comprising an amino acid sequence of SEQ ID NO: 11, SEQ ID NO: 28, or SEQ ID NO:45; and LCDR3 comprising an amino acid sequence of SEQ ID NO: 6, SEQ ID NO: 23, SEQ ID NO:40.

In another embodiment, the anti-CEA antibody or an antigen-binding fragment thereof comprises:(a) a heavy chain variable region comprising three complementarity determining regions (HCDRs) which are HCDR1 comprising an amino acid sequence of SEQ ID NO: 7, HCDR2 comprising an amino acid sequence of SEQ ID NO: 8, and HCDR3 comprising an amino acid sequence of SEQ ID NO: 9; or HCDR1 comprising an amino acid sequence of SEQ ID NO: 24, HCDR2 comprising an amino acid sequence of SEQ ID NO: 25, and HCDR3 comprising an amino acid sequence of SEQ ID NO: 26; or HCDR1 comprising an amino acid sequence of SEQ ID NO: 41, HCDR2 comprising an amino acid sequence of SEQ ID NO: 42, and HCDR3 comprising an amino acid sequence of SEQ ID NO: 43; and/or (b) a light chain variable region comprising three complementarity determining regions (LCDRs) which are LCDR1 comprising an amino acid sequence of SEQ ID NO: 10, LCDR2 comprising an amino acid sequence of SEQ ID NO: 11, and LCDR3 comprising an amino acid sequence of SEQ ID NO: 6; or LCDR1 comprising an amino acid sequence of SEQ ID NO: 27, LCDR2 comprising an amino acid sequence of SEQ ID NO: 28, and LCDR3 comprising an amino acid sequence of SEQ ID NO: 23; or LCDR1 comprising an amino acid sequence of SEQ ID NO: 44, LCDR2 comprising an amino acid sequence of SEQ ID NO: 45, and LCDR3 comprising an amino acid sequence of SEQ ID NO: 40.

In another embodiment, the anti-CEA antibody or the antigen-binding fragment thereof comprises: a heavy chain variable region that comprises (a) a HCDR1 of SEQ ID NO: 7, (b) a HCDR2 of SEQ ID NO:8, (c) a HCDR3 of SEQ ID NO:9 and a light chain variable region that comprises: (d) a LCDR1 of SEQ ID NO: 10, (e) a LCDR2 of SEQ ID NO: 11, and (f) a LCDR3 of SEQ ID NO:6.

In another embodiment, anti-CEA antibody or the antigen-binding fragment thereof comprises: a heavy chain variable region that comprises (a) a HCDR1 of SEQ ID NO:24, (b) a HCDR2 of SEQ ID NO:25, (c) a HCDR3 of SEQ ID NO:26; and a light chain variable region that comprises: (d) a LCDR1 of SEQ ID NO:27, (e) a LCDR2 of SEQ ID NO:28 and (f) a LCDR3 of SEQ ID NO:23.

In yet another embodiment, the anti-CEA antibody or the antigen-binding fragment comprises: a heavy chain variable region that comprises (a) a HCDR1 of SEQ ID NO:41, (b) a HCDR2 of SEQ ID NO:42, (c) a HCDR3 of SEQ ID NO:43; and a light chain variable region that comprises: (d) a LCDR1 of SEQ ID NO:44, (e) a LCDR2 of SEQ ID NO:45 and (f) a LCDR3 of SEQ ID NO:40.

In one embodiment, the antibody of the present disclosure or an antigen-binding fragment thereof comprises: (a) a heavy chain variable region having an amino acid sequence of SEQ ID NO: 14, SEQ ID NO: 31 or SEQ ID NO: 48 or an amino acid sequence at least 95%, 96%, 97%, 98% or 99% identical to any one of SEQ ID NO: 14, SEQ ID NO: 31 or SEQ ID NO: 48 and/or (b) a light chain variable region comprising an amino acid sequence of SEQ ID NO: 15, SEQ ID NO: 32, or SEQ ID NO: 49, or an amino acid sequence at least 95%, 96%, 97%, 98% or 99% identical to any one of SEQ ID NO: 15, SEQ ID NO: 32, or SEQ ID NO: 49.

In another embodiment, the anti-CEA antibody of the present disclosure or an antigen-binding fragment thereof comprises: (a) a heavy chain variable region comprising an amino acid sequence of SEQ ID NO: 14, SEQ ID NO: 31 or SEQ ID NO: 48 or an amino acid sequence comprising one, two, or three amino acid substitutions in the amino acid sequence of SEQ ID NO: 14, SEQ ID NO: 31 or SEQ ID NO: 48 and/or (b) a light chain variable region comprising an amino acid sequence of SEQ ID NO: 15, SEQ ID NO: 32, or SEQ ID NO: 49 or an amino acid sequence comprising one, two, three, four, or five amino acid substitutions in the amino acid of SEQ ID NO: 15, SEQ ID NO: 32, or SEQ ID NO: 49. In another embodiment, the amino acid substitutions are conservative amino acid substitutions.

In one embodiment, the antibody of the present disclosure is of IgG1, IgG2, IgG3, or IgG4 isotype. In a more specific embodiment, the antibody of the present disclosure comprises Fc domain of wild-type human IgG1 (also referred as human IgG1wt or hulgG1) or IgG2. In another embodiment, the antibody of the present disclosure comprises Fc domain of human IgG4 with S228P and/or R409K substitutions (according to EU numbering system).

In one embodiment, the anti-CEA antibody of the present disclosure binds to CEA with a binding affinity (K_{D})) of from 1×10⁻⁶ M to 1×10⁻¹⁰ M. In another embodiment, the antibody of the present disclosure binds to CEA with a binding affinity (K_{D}) of about 1×10⁻⁶ M, about 1×10⁻⁷ M, about 1×10⁻⁸ M, about 1×10−9 M or about 1×10⁻¹⁰ M.

In another embodiment, the anti-human CEA antibody of the present disclosure shows a cross-species binding activity to cynomolgus CEA.

In one embodiment, antibodies of the present disclosure have strong Fc-mediated effector functions. The antibodies mediate antibody-dependent cellular cytotoxicity (ADCC) against CEA expressing target cells.

The present disclosure relates to isolated nucleic acids comprising nucleotide sequences encoding the amino acid sequence of the anti-CEA antibody or antigen-binding fragment. In one embodiment, the isolated nucleic acid comprises a VH nucleotide sequence of SEQ ID NO: 16, SEQ ID NO: 33 or SEQ ID NO: 50 or a nucleotide sequence comprising at least 95%, 96%, 97%, 98% or 99% identity to SEQ ID NO: 16, SEQ ID NO: 33 or SEQ ID NO: 50, and encodes the VH region of the antibody or an antigen-binding fragment of the present disclosure. Alternatively or additionally, the isolated nucleic acid comprises a VL nucleotide sequence of SEQ ID NO: 17, SEQ ID NO: 34, or SEQ ID NO: 51 or a nucleotide sequence comprising at least 95%, 96%, 97%, 98% or 99% identity to SEQ ID NO: 17, SEQ ID NO: 34, or SEQ ID NO: 51, and encodes the VL region the antibody or an antigen-binding fragment of the present disclosure.

In another aspect, the present disclosure relates to a pharmaceutical composition comprising the anti-CEA antibody or antigen-binding fragment thereof, and optionally a pharmaceutically acceptable excipient.

In yet another aspect, the present disclosure relates to a method of treating a disease in a subject, which comprises administering the anti-CEA antibody or antigen-binding fragment thereof, or an anti-CEA antibody pharmaceutical composition in a therapeutically effective amount to a subject in need thereof. In another embodiment the disease to be treated by the antibody or the antigen-binding fragment is cancer.

The current disclosure relates to use of the anti-CEA antibody or the antigen-binding fragment thereof, or an anti-CEA antibody pharmaceutical composition for treating a disease, such as cancer.

### BRIEF DESCRIPTION OF THE DRAWINGS

FIG. 1 shows schematic diagrams of shed CEA (sCEA), chimeric CEA (CHIM), CEACAM6 and CEA variants (CEA-v). In CEA, domains N, A1, B1, A2, B2, A3, B3 and GPI linker (GPI) are labeled; in CEACAM6, domains N′, A′ and B′ are labeled.

FIG. 2A-B depicts phylogenetic trees of anti-CEA domain B3 antibody VH (FIG. 2A) and VL (FIG. 2B) regions. The VH and VL sequences of candidate anti-CEA antibodies were aligned using DNASTAR's Megalign™ software. Sequence homology was displayed in phylogenetic trees.

FIG. 3A shows affinity determination of purified murine anti-CEA antibody BGA13 on a chimeric construct (CHIM) by surface plasmon resonance (SPR). FIG. 3B depicts binding profiles of BGA13 by antigen ELISA.

FIG. 4A-B shows effects of soluble CEA (sCEA) on CEA antibodies binding to MKN45 cells. FIG. 4A shows binding profiles of domain B3 antibodies in the presence or absence of soluble CEA (sCEA); FIG. 4B are the antibody binding profiles of FIG. 4A shown as a histogram.

FIG. 5A-B shows the randomization sites for generating an antibody library for affinity maturation of humanized BGA13 antibody light chain CDR (LCDR) regions (FIG. 5A) and heavy chain CDR (HCDR) regions (FIG. 5B).

FIG. 6 shows the amino acid changes of BGA13 light chain CDR regions after four rounds of selection.

FIG. 7 shows the binding to LOVO cells of affinity matured, humanized BGA13 variants by flow cytometry.

FIG. 8 is the binding to MKN45 cells of optimized, humanized BGA113 variants by flow cytometry.

FIGS. 9A and 9B demonstrate that there is no off-target binding of antibody BGA113K to various CEACAM family members by flow cytometry (FIG. 9A) and antigen ELISA (FIG. 9B).

FIG. 10 show the effects of soluble CEA on BGA113K binding to CEA expressing cells MKN45 cells in the presence of various concentrations of soluble CEA.

FIG. 11 demonstrates that antibody BGA113 kills cells by ADCC in vitro.

FIG. 12 depicts the reduction in tumor volume of a murine cancer model when treated with BGA113 antibody.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “Carcinoembryonic antigen” or “CEA” refers to an approximately 70-100 kDa glycoprotein. CEA is also known as CEACAM5 or CD66e. The amino acid sequence of human CEA, (SEQ ID NO: 52) can also be found at accession number P06731 or NM_004363.2.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-CEA antibodies comprise at least one antigen-binding site, at least a variable region. In some embodiments, the anti-CEA antibodies comprise an antigen-binding fragment from an CEA antibody described herein. In some embodiments, the anti-CEA antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as a, 8, &, Y, or u, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); A1-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to CEA. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}: C_{H}I interfaces or V_{H}/V_{L}. interfaces of antibodies (see, e.g., US 2011/0287009, US2007/0178552, WO 96/027011, WO 98/050431, and Zhu et al., 1997, Protein Science 6:781-788). In some embodiments, knob-into-holes insure the correct pairing of two different heavy chains together during the manufacture of antibodies. For example, antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-CEA antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CEA antibody is administered to the subject at the same time as, just before, or just after administration of an additional therapeutic agent. In certain embodiments, an anti-CEA antibody is administered as a co-formulation with an additional therapeutic agent.

The term “toxin” or “payload” or “cytotoxic agent” is used herein as a molecule that inhibits or reduces the expression of molecules in cells, function of cells, induces apoptosis of cells and/or causes destruction of cells. The term includes radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to, auristatins (e.g., auristatin E, auristatin F, MMAE and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin as well as radioisotopes such as At211, I131, 1125, Y90, Re186, Re188, Sm153, Bi212 or 213, P32, and Lu177.

### DETAILED DESCRIPTION

The present disclosure provides for anti-CEA antibodies and antigen-binding fragments thereof. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### Anti-CEA Antibodies

The present disclosure provides for antibodies or antigen-binding fragments thereof that specifically bind to CEA. Antibodies or antigen-binding fragments of the present disclosure include, but are not limited to, the antibodies or antigen-binding fragments thereof, generated as described, below.

The present disclosure provides antibodies or antigen-binding fragments that specifically bind to CEA, wherein said antibodies or antibody fragments (e.g., antigen-binding fragments) comprise a VH domain having an amino acid sequence of SEQ ID NOs: 14, 31 or 48 (Table 1). The present disclosure also provides antibodies or antigen-binding fragments that specifically bind CEA, wherein said antibodies or antigen-binding fragments comprise a HCDR having an amino acid sequence of any one of the HCDRs listed in Table 1. In one aspect, the present disclosure provides antibodies or antigen-binding fragments that specifically bind to CEA, wherein said antibodies comprise (or alternatively, consist of) one, two, three, or more HCDRs having an amino acid sequence of any of the HCDRs listed in Table 1.

The present disclosure provides for antibodies or antigen-binding fragments that specifically bind to CEA, wherein said antibodies or antigen-binding fragments comprise a VL domain having an amino acid sequence of SEQ ID NO: 15, 32 or 49 (Table 1). The present disclosure also provides antibodies or antigen-binding fragments that specifically bind to CEA, wherein said antibodies or antigen-binding fragments comprise a LCDR having an amino acid sequence of any one of the LCDRs listed in Table 1. In particular, the disclosure provides for antibodies or antigen-binding fragments that specifically bind to CEA, said antibodies or antigen-binding fragments comprise (or alternatively, consist of) one, two, three or more LCDRs having an amino acid sequence of any of the LCDRs listed in Table 1.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH, VL, the full length heavy chain, and the full length light chain of the antibodies that specifically bind to CEA. Such nucleic acid sequences can be optimized for expression in mammalian cells.

TABLE 1

| ANTIBODY | SEQ ID NO | | SEQUENCE |
|---|---|---|---|
| BGA113K | SEQ ID NO: 1 | HCDR1 | GYIFTSYY |
| (IMGT) | | (IMGT) | |
| | SEQ ID NO: 2 | HCDR2 | INPQTGKT |
| | | (IMGT) | |
| | SEQ ID NO: 3 | HCDR3 | AREYGNYNYPLDY |
| | | (IMGT) | |
| | SEQ ID NO: 4 | LCDR1 | ENQYGY |
| | | (IMGT) | |
| | SEQ ID NO: 5 | LCDR2 | NY |
| | | (IMGT) | |
| | SEQ ID NO: 6 | LCDR3 | QHHLGTPYT |
| | | (IMGT) | |
| BGA113K | SEQ ID NO: 7 | HCDR1 | SYYLH |
| (Kabat) | | (Kabat) | |
| | SEQ ID NO: 8 | HCDR2 | YINPQTGKTSYAQKFQG |
| | | (Kabat) | |
| | SEQ ID NO: 9 | HCDR3 | EYGNYNYPLDY |
| | | (Kabat) | |
| | SEQ ID NO: | LCDR1 | RASENQYGYLA |
| | 10 | (Kabat) | |
| | SEQ ID NO: | LCDR2 | NYKNLVE |
| | 11 | (Kabat) | |
| | SEQ ID NO: 6 | LCDR3 | QHHLGTPYT |
| | | (Kabat) | |
| BGA113K | SEQ ID NO: | HCDR1 | GYIFTSY |
| (Chothia) | 12 | (Chothia) | |
| | SEQ ID NO: | HCDR2 | NPQTGK |
| | 13 | (Chothia) | |
| | SEQ ID NO: 9 | HCDR3 | EYGNYNYPLDY |
| | | (Chothia) | |
| | SEQ ID NO: | LCDR1 | RASENQYGYLA |
| | 10 | (Chothia) | |
| | SEQ ID NO: | LCDR2 | NYKNLVE |
| | 11 | (Chothia) | |
| | SEQ ID NO: 6 | LCDR3 | QHHLGTPYT |
| | | (Chothia) | |
| BGA113K | SEQ ID NO: | VH | QVQLVQSGAEVKKPGASVKVSCKASGYIFTSY |
| | 14 | | YLHWVRQAPGQGLEWIGYINPQTGKTSYAQK |
| | | | FQGRVTMTRDTSTSTVYMELSSLRSEDTAVYY |
| | | | CAREYGNYNYPLDYWGQGTLVTVSS |
| | SEQ ID NO: | VL | DIQMTQSPSSLSASVGDRVTITCRASENQYGYL |
| | 15 | | AWYQQKPGKVPKLLIYNYKNLVEGVPSRFSGS |
| | | | GSGTDFTLTISSLQPEDVATYYCQHHLGTPYTF |
| | | | GQGTKVEIK |
| | SEQ ID NO: | VH DNA | CAGGTGCAGCTGGTGCAGAGCGGCGCGGAA |
| | 16 | | GTGAAAAAACCGGGCGCGAGCGTGAAAGTG |
| | | | AGCTGCAAAGCGAGCGGCTATATTTTTACCA |
| | | | GCTATTACCTGCATTGGGTGCGCCAGGCGCC |
| | | | GGGCCAGGGCCTGGAATGGATTGGCTATATTA |
| | | | ACCCGCAGACCGGCAAGACCAGCTATGCCCA |
| | | | GAAATTTCAGGGCCGCGTGACCATGACCCGC |
| | | | GATACCAGCACCAGCACCGTGTATATGGAAC |
| | | | TGAGCAGCCTGCGCAGCGAAGATACCGCGGT |
| | | | GTATTATTGCGCGCGCGAATATGGCAACTATA |
| | | | ACTATCCGCTGGATTATTGGGGCCAGGGCAC |
| | | | CCTGGTGACCGTGAGCAGC |
| | SEQ ID NO: | VL DNA | GATATTCAGATGACCCAGAGCCCGAGCAGCC |
| | 17 | | TGAGCGCGAGCGTGGGCGATCGCGTGACCAT |
| | | | TACCTGCCGCGCGAGCGAAAACCAGTATGGC |
| | | | TATCTGGCGTGGTATCAGCAGAAACCGGGCA |
| | | | AAGTGCCGAAACTGCTGATTTATAACTATAAA |
| | | | AACCTGGTGGAAGGCGTGCCGAGCCGCTTTA |
| | | | GCGGCAGCGGCAGCGGCACCGATTTTACCCT |
| | | | GACCATTAGCAGCCTGCAGCCGGAAGATGTG |
| | | | GCGACCTATTATTGCCAGCATCATCTGGGCAC |
| | | | CCCGTATACCTTTGGCCAGGGCACCAAAGTG |
| | | | GAAATTAAA |
| BGA190 | SEQ ID NO: | HCDR1 | GFTFSSFG |
| (IMGT) | 18 | (IMGT) | |
| | SEQ ID NO: | HCDR2 | ISIGSDII |
| | 19 | (IMGT) | |
| | SEQ ID NO: | HCDR3 | TRRSYRSYWYFDV |
| | 20 | (IMGT) | |
| | SEQ ID NO: | LCDR1 | ENIYSY |
| | 21 | (IMGT) | |
| | SEQ ID NO: | LCDR2 | NA |
| | 22 | (IMGT) | |
| | SEQ ID NO: | LCDR3 | QHHFLSPWM |
| | 23 | (IMGT) | |
| BGA190 | SEQ ID NO: | HCDR1 | SFGMH |
| (Kabat) | 24 | (Kabat) | |
| | SEQ ID NO: | HCDR2 | YISIGSDIIYYADTVKG |
| | 25 | (Kabat) | |
| | SEQ ID NO: | HCDR3 | RSYRSYWYFDV |
| | 26 | (Kabat) | |
| | SEQ ID NO: | LCDR1 | RTSENIYSYLA |
| | 27 | (Kabat) | |
| | SEQ ID NO: | LCDR2 | NAKTLAE |
| | 28 | (Kabat) | |
| | SEQ ID NO: | LCDR3 | QHHFLSPWM |
| | 23 | (Kabat) | |
| BGA190 | SEQ ID NO: | HCDR1 | GFTFSSF |
| (Chothia) | 29 | (Chothia) | |
| | SEQ ID NO: | HCDR2 | SIGSDI |
| | 30 | (Chothia) | |
| | SEQ ID NO: | HCDR3 | RSYRSYWYFDV |
| | 26 | (Chothia) | |
| | SEQ ID NO: | LCDR1 | RTSENIYSYLA |
| | 27 | (Chothia) | |
| | SEQ ID NO: | LCDR2 | NAKTLAE |
| | 28 | (Chothia) | |
| | SEQ ID NO: | LCDR3 | QHHFLSPWM |
| | 23 | (Chothia) | |
| BGA190 | SEQ ID NO: | VH | DVQLVESGGGLVQPGGSRELSCAASGFTFSSFG |
| | 31 | | MHWVRQAPERGLEWVAYISIGSDIIYYADTVK |
| | | | GRFTISRDNPKNTLFLQMTSLRSEDTAVYYCTR |
| | | | RSYRSYWYFDVWGAGTTVTVSS |
| | SEQ ID NO: | VL | DIQMTQSPASLSASVGETVTITCRTSENIYSYLA |
| | 32 | | WYQQKQGKSPHLLVYNAKTLAEGVPSRFSGS |
| | | | GSGTQFSLKIISLQPEDFGTYYCQHHFLSPWMF |
| | | | GGGTKLEIK |
| | SEQ ID NO: | VH DNA | GATGTGCAGCTGGTGGAGTCTGGGGGAGGCT |
| | 33 | | TAGTGCAGCCTGGAGGGTCCCGGGAACTCTC |
| | | | CTGTGCAGCCTCTGGATTCACTTTCAGTAGCT |
| | | | TTGGAATGCACTGGGTTCGTCAGGCTCCAGA |
| | | | GAGGGGGCTGGAGTGGGTCGCATACATTAGT |
| | | | ATTGGCAGTGATATCATCTACTATGCAGACAC |
| | | | AGTGAAGGGCCGATTCACCATCTCCAGAGAC |
| | | | AATCCCAAGAACACCCTGTTCCTGCAAATGA |
| | | | CCAGTCTGAGGTCTGAGGACACGGCCGTGTA |
| | | | TTACTGTACAAGAAGGTCTTATAGGTCCTACT |
| | | | GGTACTTCGATGTCTGGGGCGCAGGGACCAC |
| | | | GGTCACCGTCTCCTCA |
| | SEQ ID NO: | VL DNA | GACATCCAGATGACTCAGTCTCCAGCCTCCC |
| | 34 | | TATCTGCATCTGTGGGAGAAACTGTCACCATC |
| | | | ACATGTCGAACAAGTGAGAATATTTACAGTT |
| | | | ATTTAGCATGGTATCAGCAGAAACAGGGAAA |
| | | | ATCTCCTCATCTCCTGGTCTATAATGCAAAAA |
| | | | CCTTAGCAGAGGGTGTGCCATCAAGGTTCAG |
| | | | TGGCAGTGGATCAGGCACACAGTTTTCTCTG |
| | | | AAGATCATCAGCCTGCAGCCTGAAGATTTTG |
| | | | GGACTTATTACTGTCAGCATCATTTTCTTAGT |
| | | | CCGTGGATGTTCGGTGGAGGCACCAAGCTGG |
| | | | AAATCAAA |
| BGA288 | SEQ ID NO: | HCDR1 | GYTFTDYN |
| (IMGT) | 35 | (IMGT) | |
| | SEQ ID NO: | HCDR2 | IGPNYGGT |
| | 36 | (IMGT) | |
| | SEQ ID NO: | HCDR3 | ARRGSIPRAVDY |
| | 37 | (IMGT) | |
| | SEQ ID NO: | LCDR1 | QDIHNY |
| | 38 | (IMGT) | |
| | SEQ ID NO: | LCDR2 | RA |
| | 39 | (IMGT) | |
| | SEQ ID NO: | LCDR3 | LQYDEFPYT |
| | 40 | (IMGT) | |
| BGA288 | SEQ ID NO: | HCDR1 | DYNID |
| (Kabat) | 41 | (Kabat) | |
| | SEQ ID NO: | HCDR2 | DIGPNYGGTIYNQKFKG |
| | 42 | (Kabat) | |
| | SEQ ID NO: | HCDR3 | RGSIPRAVDY |
| | 43 | (Kabat) | |
| | SEQ ID NO: | LCDR1 | KASQDIHNYLS |
| | 44 | (Kabat) | |
| | SEQ ID NO: | LCDR2 | RANRLVD |
| | 45 | (Kabat) | |
| | SEQ ID NO: | LCDR3 | LQYDEFPYT |
| | 40 | (Kabat) | |
| BGA288 | SEQ ID NO: | HCDR1 | GYTFTDY |
| (Chothia) | 46 | (Chothia) | |
| | SEQ ID NO: | HCDR2 | GPNYGG |
| | 47 | (Chothia) | |
| | SEQ ID NO: | HCDR3 | RGSIPRAVDY |
| | 43 | (Chothia) | |
| | SEQ ID NO: | LCDR1 | KASQDIHNYLS |
| | 44 | (Chothia) | |
| | SEQ ID NO: | LCDR2 | RANRLVD |
| | 45 | (Chothia) | |
| | SEQ ID NO: | LCDR3 | LQYDEFPYT |
| | 40 | (Chothia) | |
| BGA288 | SEQ ID NO: | VH | EVLLQQSGPELVKPGASVKIFCKASGYTFTDY |
| | 48 | | NIDWVQQSHGKSLEWIGDIGPNYGGTIYNQKF |
| | | | KGKATLTVAKSSSTAYMELRSLTSEDTAVYYCA |
| | | | RRGSIPRAVDYWGQGTSVTVSS |
| | SEQ ID NO: | VL | DIKMTQSPSSMYASLGERVTITCKASQDIHNYL |
| | 49 | | SWFQQKPGKSPKTLIYRANRLVDGVPSRFSGS |
| | | | GSGQDYSLTISSLEYEDMGIYYCLQYDEFPYTF |
| | | | GGGTKLEIK |
| | SEQ ID NO: | VH DNA | GAGGTCCTGCTGCAACAGTCTGGACCTGAGC |
| | 50 | | TGGTGAAGCCTGGGGCTTCAGTGAAGATTTT |
| | | | CTGTAAGGCTTCTGGATACACATTCACTGACT |
| | | | ACAACATAGACTGGGTGCAGCAGAGCCATGG |
| | | | AAAGAGCCTTGAGTGGATTGGAGATATTGGT |
| | | | CCTAATTATGGTGGTACTATCTACAACCAGAA |
| | | | GTTCAAGGGCAAGGCCACATTGACTGTAGCC |
| | | | AAGTCCTCCAGCACAGCCTACATGGAGCTCC |
| | | | GCAGCCTGACATCTGAGGACACTGCAGTCTA |
| | | | TTACTGTGCAAGACGCGGTAGCATCCCGAGG |
| | | | GCTGTGGACTACTGGGGTCAAGGAACCTCAG |
| | | | TCACCGTCTCCTCA |
| | SEQ ID NO: | VL DNA | GACATCAAGATGACCCAGTCTCCATCTTCCAT |
| | 51 | | GTATGCATCTCTAGGAGAGAGAGTCACTATC |
| | | | ACTTGCAAGGCGAGTCAGGACATTCATAACT |
| | | | ATTTAAGCTGGTTCCAGCAGAAACCAGGGAA |
| | | | ATCTCCTAAGACCCTGATCTATCGTGCAAACA |
| | | | GATTGGTAGATGGGGTCCCATCAAGGTTCAG |
| | | | TGGCAGTGGATCTGGGCAAGATTATTCTCTCA |
| | | | CCATCAGCAGCCTGGAGTATGAAGATATGGG |
| | | | AATTTATTATTGTCTACAGTATGATGAGTTTCC |
| | | | GTACACGTTCGGAGGGGGGACCAAGCTGGA |
| | | | AATAAAA |

The present disclosure provides antibodies and antigen-binding fragments thereof that bind to an epitope of human CEA. In certain aspects the antibodies and antigen-binding fragments can bind to the same epitope of CEA.

The present disclosure also provides for antibodies and antigen-binding fragments thereof that bind to the same epitope as do the anti-CEA antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CEA demonstrates that the test antibody can compete with that antibody or antigen-binding fragments thereof for binding to CEA. Such an antibody can, without being bound to any one theory, bind to the same or a related (e.g., a structurally similar or spatially proximal) epitope on CEA as the antibody or antigen-binding fragments thereof with which it competes. In a certain aspect, the antibody that binds to the same epitope on CEA as the antibodies or antigen-binding fragments thereof of the present disclosure is a human or humanized monoclonal antibody. Such human or humanized monoclonal antibodies can be prepared and isolated as described herein.

### Linkers

It is also understood that the domains and/or regions of the polypeptide chains of the antibody can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1 Such linker region may comprise a random assortment of amino acids, or a restricted set of amino acids. Such linker regions can be flexible or rigid (see US2009/0155275).

In different embodiments linkers can be used to conjugate compounds between a toxin and the antibody, in some embodiments, the linker is cleavable under intracellular conditions, such that cleavage of the linker releases the toxin from the antibody in the intracellular environment. In yet other embodiments, the linker unit is not cleavable and the toxin is released, for example, by antibody degradation. The linker can be without limitation, a cleavable linker, a non-cleavable linker, a hydrophilic linker, a procharged linker and a dicarboxylic acid based linker.

### Dimerization Specific Amino Acids

In one embodiment, the antibody comprises at least one dimerization specific amino acid change. The dimerization specific amino acid changes result in “knobs into holes” interactions, and increases the assembly of correct antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551. The dimerization specific amino acids can also be within the Fc domain and can be in combination with dimerization specific amino acids within the CH1 or CL domains. In one embodiment, the disclosure provides an antibody comprising at least one dimerization specific amino acid pair.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered Clq binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of the antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or Clq binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev Immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 16, SEQ ID NO: 33 or SEQ ID NO: 50. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 17, 34, or 51.

The polynucleotides of the present disclosure can encode the variable region sequence of an anti-CEA antibody. They can also encode both a variable region and a constant region of the antibody. Some of the polynucleotide sequences encode a polypeptide that comprises variable regions of both the heavy chain and the light chain of the exemplified anti-CEA antibodies.

Also provided in the present disclosure are expression vectors and host cells for producing the anti-CEA antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CEA antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CEA antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CEA antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CEA antibodies. Insect cells in combination with baculovirus vectors can also be used.

In other aspects, mammalian host cells are used to express and produce the anti-CEA antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of CEA. In one aspect, the antibodies or antigen-binding fragments are useful for detecting the presence of CEA in a biological sample. The term “detecting” as used herein includes quantitative or qualitative detection. In certain aspects, a biological sample comprises a cell or tissue. In other aspects, such tissues include normal and/or cancerous tissues that express CEA at higher levels relative to other tissues.

In one aspect, the present disclosure provides a method of detecting the presence of CEA in a biological sample. In certain aspects, the method comprises contacting the biological sample with an anti-CEA antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of CEA. In certain aspects, the method comprises contacting a test cell with an anti-CEA antibody; determining the level of expression (either quantitatively or qualitatively) of CEA expressed by the test cell by detecting binding of the anti-CEA antibody to the CEA polypeptide; and comparing the level of expression by the test cell with the level of CEA expression in a control cell (e.g., a normal cell of the same tissue origin as the test cell or a non-CEA expressing cell), wherein a higher level of CEA expression in the test cell as compared to the control cell indicates the presence of a disorder associated with expression of CEA.

### Methods of Treatment

The antibodies or antigen-binding fragments of the present disclosure are useful in a variety of applications including, but not limited to, methods for the treatment of a CEA-associated disorder or disease. In one aspect, the CEA-associated disorder or disease is a cancer.

In one aspect, the present disclosure provides a method of treating cancer. In certain aspects, the method comprises administering to a patient in need an effective amount of an anti-CEA antibody or antigen-binding fragment. The cancer can include, without limitation, gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma and sarcoma.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g., every week or every three weeks (e.g., such that the patient receives from about two to about twenty, or e.g., about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, anti-CEA antibodies of the present disclosure can be used in combination with other therapeutic agents. Other therapeutic agents that can be used with the anti-CEA antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

In another aspect, the anti-CEA antibodies can be used in combination with an anti-PD1 antibody. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), as disclosed by Merck, in U.S. Pat. Nos. 8,354,509 and 8,900,587 is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Pharmaceutical Compositions and Formulations

Also provided are compositions, including pharmaceutical formulations, comprising an anti-CEA antibody or antigen-binding fragment thereof, or polynucleotides comprising sequences encoding an anti-CEA antibody or antigen-binding fragment. In certain embodiments, compositions comprise one or more anti-CEA antibodies or antigen-binding fragments, or one or more polynucleotides comprising sequences encoding one or more anti-CEA antibodies or antigen-binding fragments. These compositions can further comprise suitable carriers, such as pharmaceutically acceptable excipients including buffers, which are well known in the art.

Pharmaceutical formulations of an anti-CEA antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### EXAMPLES

### Example 1: Generation of Anti-CEA Monoclonal Antibody

### CEA Recombinant Proteins for Immunization and Binding Assays

To discover new antibodies against CEA that both cross-react with human and Macaca mulatta CEA in the membrane peripheral region containing domain B3, (amino acids 596-674 of SEQ ID NO:52, see Beauchemin et al., Mol. Cell Bio., 1987, 7(9):3321-3330)). but without off-target binding with other human CEACAM members, several recombinant proteins were designed and expressed for antibody screening (see Table 2).

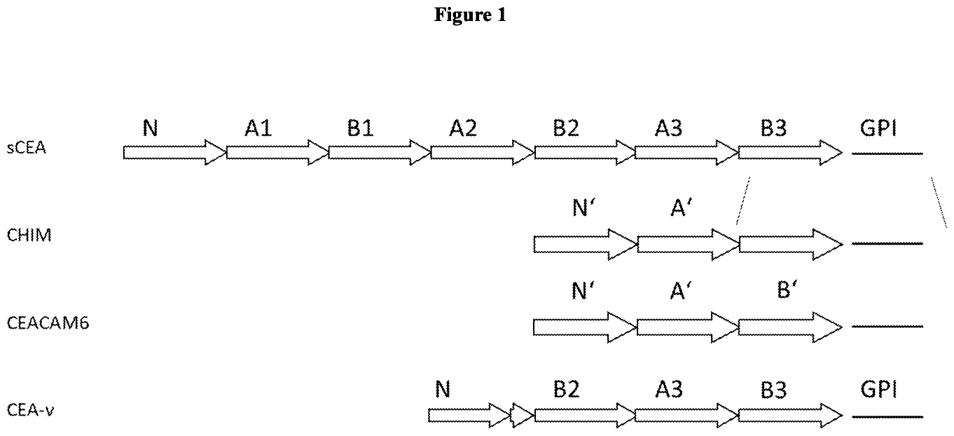

The cDNA coding regions for the full-length human CEA (SEQ ID NO: 52), Macaca CEA (SEQ ID NO: 53) and the full-length human CEACAM6 (SEQ ID NO: 54) were ordered based on the GenBank sequence. For human CEA (Accession No: NM_004363.2), the gene is available from Sinobio, Cat. No. HG11077-UT. For Macaca CEA (Accession No: NM_001047125), the gene is available from Genscript, Cat. No. OMb23865D. For human CEACAM6 (Accession No: NM_002483.4), the gene is available from Sinobio, Cat. No. HG10823-UT. The schematic presentation of CEA fusion proteins is shown in FIG. 1. It is reported a splice variant of human CEA is expressed concomitantly with full-length CEA on tumors (Peng et al., PloS one, 7, e36412-e36412 (2012)), and the variant (CEA-v) was prepared accordingly. To generate this construct, the coding region of extracellular domain (ECD) consisting of amino acid (AA) 1-687 of huCEA (SEQ ID NO: 55), the region of amino acid (AA) 1-690 of monkeyCEA (SEQ ID NO: 56) and the region of amino acid (AA) 1-320 of CEACAM6 (SEQ ID NO:57) were PCR-amplified. The regions of CEA amino acid (AA) 1-78 (SEQ ID NO:58) and amino acids 398-687 of CEA (SEQ ID NO:59) were PCR-amplified, and then conjugated by overlap-PCR to make a CEA variant (CEA-v) (SEQ ID NO: 60). Alternatively, the regions of CEACAM6 amino acid (AA) 1-273 (SEQ ID NO: 61), and the membrane-peripheral region containing domain B3 of CEA amino acid (AA) 596-687 of (SEQ ID NO: 62) were PCR-amplified, and then conjugated by overlap-PCR to make a chimeric construct (CHIM) (SEQ ID NO: 63). All constructs were then cloned into a pcDNA3.1-based expression vector (Invitrogen, Carlsbad, CA, USA) with C-terminus fused with 6×His tags individually, which resulted in five recombinant fusion protein expression plasmids, CEA, monkeyCEA, CEACAM6, CEA-v and CHIM. For recombinant fusion protein production, CEA, monkeyCEA, CEACAM6, CEA-v and CHIM plasmids were transiently transfected into a HEK293-based mammalian cell expression system (generated in house) and cultured for 5-7 days in a CO₂ incubator equipped with a rotating shaker. The supernatants containing the recombinant proteins were collected and cleared by centrifugation. Recombinant proteins were purified using a Ni-NTA agarose (Cat. No. R90115, Invitrogen). All recombinant proteins were dialyzed against phosphate buffered saline (PBS) and stored in −80° C. freezer in small aliquots.

### Stable Expression in Cell Lines

To establish stable cell lines that express full-length human CEA (Accession No: NM_004363.2, the cDNA expressing CEA was cloned into a retroviral vector pFB-Neo (Cat. No. 217561, Agilent, USA). Dual-tropic retroviral vectors were generated according to a previous protocol (Zhang et al., Blood. 2005 106(5):1544-51). Viral vectors containing human CEA were transduced into L929 (ATCC, Manassas, VA, USA) and CT26 cells (ATCC, Manassas, VA, USA), in order to generate human CEA expressing cell lines. The high expression cell lines were selected by culture in complete RPMI1640 medium containing 10% FBS with G418, and then verified via FACS binding assay.

### Immunization, Hybridoma Fusion and Cloning

Eight to twelve week-old Balb/c mice (HFK BIOSCIENCE CO., LTD, Beijing, China) were immunized intraperitoneally (i.p.) with 500 μl of 1×10⁷ L929/huCEA cells with or without a water-soluble adjuvant (Cat. No. KX0210041, KangBiQuan, Beijing, China). The procedure was repeated two weeks later in order to boost antibody production. Two weeks after the third immunization, mouse sera were evaluated for soluble CEA (sCEA) binding by ELISA and FACS. Splenocytes were isolated and fused to the murine myeloma cell line, SP2/0 cells (ATCC, Manassas, VA, USA), using the standard techniques (Colligan J E, et al., CURRENT PROTOCOLS IN IMMUNOLOGY, 1993).

### Assessment of CEA Binding Activity of Antibodies by ELISA and FACS

To screen for antibodies that bound human CEA, but did not bind CEACAM6 or sCEA, antibodies which bound to CHIM but not to sCEA, CEACAM6 and CEA-v, and antibodies which bind to CHIM, sCEA and CEA-v, but not to CEACAM6 were screened and counter-screened. The supernatants of hybridoma clones were initially screened by ELISA as described in (Methods in Molecular Biology (2007) 378:33-52) with some modifications. Briefly, sCEA, CHIM, CEACAM6 or CEA-v were coated in 96-well plates at a low concentration of 3 μg/ml, individually. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm™, Molecular Devices, USA). The ELISA-positive clones were further verified by FACS using either the L929/huCEA and/or MKN45 cells (ATCC). MKN45 cells are of human gastric cancer origin. CEA-expressing cells (10⁵ cells/well) were incubated with ELISA-positive hybridoma supernatants, followed by binding with Alexa Fluro-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA).

The conditioned media from the hybridomas that showed positive signals in FACS screening, and binding to CHIM but not CEACAM6 and sCEA were subjected to functional assays to evaluate the presence of sCEA on the binding of CEA antibodies to CEA expressing cells (see the Examples below). The antibodies with the desired binding specificity and functional activities were further sub-cloned and characterized.

### Subcloning and Adaptation of Hybridomas to Serum-Free or Low Serum Medium

After screening primarily by ELISA, FACS and functional assays, the positive hybridoma clones were sub-cloned by limiting dilution. The top antibody subclones verified through functional assays were adapted for growth in the CDM4MAb medium (Cat. No. SH30801.02, Hyclone, USA) with 3% FBS.

### Expression and Purification of Monoclonal Antibodies

Hybridoma cells were cultured in CDM4MAb medium (Cat. No. SH30801.02, Hyclone), and incubated in a CO₂ incubator for 5 to 7 days at 37° C. The conditioned medium was collected through centrifugation and filtration by passing through a 0.22 μm membrane before purification. Murine antibody-containing supernatants were applied and bound to a Protein A column (Cat. No. 17127901, GE Life Sciences) following the protocol in the manufacturer's guide. The procedure usually yielded antibodies at purity above 90%. The Protein A-affinity purified antibodies were either dialyzed against PBS or further purified using a HiLoad 16/60 Superdex™ 200 column (Cat. No. 17531801, GE Life Sciences) to remove aggregates. Protein concentrations were determined by measuring absorbance at 280 nm. The final antibody preparations were stored in aliquots in −80° C. freezer.

TABLE 2
amino acid and nucleic acid sequences

| SEQ ID NO: | Construct | SEQUENCE |
|---|---|---|
| SEQ ID | full-length | MESPSAPPHRWCIPWQRLLLTASLLTFWNPPTTAKLTIESTPFNV |
| NO: 52 | human | AEGKEVLLLVHNLPQHLFGYSWYKGERVDGNRQIIGYVIGTQQ |
| | CEA | ATPGPAYSGREIIYPNASLLIQNIIQNDTGFYTLHVIKSDLVNEEA |
| | | TGQFRVYPELPKPSISSNNSKPVEDKDAVAFTCEPETQDATYLW |
| | | WVNNQSLPVSPRLQLSNGNRTLTLFNVTRNDTASYKCETQNPV |
| | | SARRSDSVILNVLYGPDAPTISPLNTSYRSGENLNLSCHAASNPP |
| | | AQYSWFVNGTFQQSTQELFIPNITVNNSGSYTCQAHNSDTGLN |
| | | RTTVTTITVYAEPPKPFITSNNSNPVEDEDAVALTCEPEIQNTTYL |
| | | WWVNNQSLPVSPRLQLSNDNRTLTLLSVTRNDVGPYECGIQNE |
| | | LSVDHSDPVILNVLYGPDDPTISPSYTYYRPGVNLSLSCHAASN |
| | | PPAQYSWLIDGNIQQHTQELFISNITEKNSGLYTCQANNSASGH |
| | | SRTTVKTITVSAELPKPSISSNNSKPVEDKDAVAFTCEPEAQNTT |
| | | YLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGATVGIMIGVLVGVALI |
| SEQ ID | Macaca | MGSPSAPLHRWCIPWQTLLLTASLLTFWNPPTTAQLTIESRPFNV |
| NO: 53 | CEA | AEGKEVLLLAHNVSQNLFGYIWYKGERVDASRRIGSCVIRTQQI |
| | | TPGPAHSGRETIDFNASLLIHNVTQSDTGSYTIQVIKEDLVNEEA |
| | | TGQFRVYPELPKPYISSNNSNPVEDKDAVALTCEPETQDTTYLW |
| | | WVNNQSLPVSPRLELSSDNRTLTVFNIPRNDTTSYKCETQNPVS |
| | | VRRSDPVTLNVLYGPDAPTISPLNTPYRAGENLNLTCHAASNPT |
| | | AQYFWFVNGTFQQSTQELFIPNITVNNSGSYMCQAHNSATGLN |
| | | RTTVTAITVYAELPKPYITSNNSNPIEDKDAVTLTCEPETQDTTY |
| | | LWWVNNQSLSVSSRLELSNDNRTLTVFNIPRNDTTFYECETQNP |
| | | VSVRRSDPVTLNVLYGPDAPTISPLNTPYRAGENLNLSCHAASN |
| | | PAAQYSWFVNGTFQQSTQELFIPNITVNNSGSYMCQAHNSATG |
| | | LNRTTVTAITVYVELPKPYISSNNSNPIEDKDAVTLTCEPVAENT |
| | | TYLWWVNNQSLSVSPRLQLSNGNRILTLLSVTRNDTGPYECGI |
| | | QNSESAKRSDPVTLNVTYGPDTPIISPPDLSYRSGANLNLSCHS |
| | | DSNPSPQYSWLINGTLRQHTQVLFISKITSNNSGAYACFVSNLAT |
| | | GRNNSIVKNISVSSGDSAPGSSGLSARATVGIIIGMLVGVALM |
| SEQ ID | full-length | MGPPSAPPCRLHVPWKEVLLTASLLTFWNPPTTAKLTIESTPFNV |
| NO: 54 | human | AEGKEVLLLAHNLPQNRIGYSWYKGERVDGNSLIVGYVIGTQQ |
| | CEACAM | ATPGPAYSGRETIYPNASLLIQNVTQNDTGFYTLQVIKSDLVNEE |
| | 6 | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPSKANYRPGENLNLSCHAAS |
| | | NPPAQYSWFINGTFQQSTQELFIPNITVNNSGSYMCQAHNSATG |
| | | LNRTTVTMITVSGSAPVLSAVATVGITIGVLARVALI |
| SEQ ID | 1-687 of | MESPSAPPHRWCIPWQRLLLTASLLTFWNPPTTAKLTIESTPFNV |
| NO: 55 | huCEA | AEGKEVLLLVHNLPQHLFGYSWYKGERVDGNRQIIGYVIGTQQ |
| | | ATPGPAYSGREIIYPNASLLIQNIIQNDTGFYTLHVIKSDLVNEEA |
| | | TGQFRVYPELPKPSISSNNSKPVEDKDAVAFTCEPETQDATYLW |
| | | WVNNQSLPVSPRLQLSNGNRTLTLFNVTRNDTASYKCETQNPV |
| | | SARRSDSVILNVLYGPDAPTISPLNTSYRSGENLNLSCHAASNPP |
| | | AQYSWFVNGTFQQSTQELFIPNITVNNSGSYTCQAHNSDTGLN |
| | | RTTVTTITVYAEPPKPFITSNNSNPVEDEDAVALTCEPEIQNTTYL |
| | | WWVNNQSLPVSPRLQLSNDNRTLTLLSVTRNDVGPYECGIQNE |
| | | LSVDHSDPVILNVLYGPDDPTISPSYTYYRPGVNLSLSCHAASN |
| | | PPAQYSWLIDGNIQQHTQELFISNITEKNSGLYTCQANNSASGH |
| | | SRTTVKTITVSAELPKPSISSNNSKPVEDKDAVAFTCEPEAQNTT |
| | | YLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGAHHHHHH |
| SEQ ID | 1-690 of | MGSPSAPLHRWCIPWQTLLLTASLLTFWNPPTTAQLTIESRPFNV |
| NO: 56 | monkey | AEGKEVLLLAHNVSQNLFGYIWYKGERVDASRRIGSCVIRTQQI |
| | CEA | TPGPAHSGRETIDFNASLLIHNVTQSDTGSYTIQVIKEDLVNEEA |
| | | TGQFRVYPELPKPYISSNNSNPVEDKDAVALTCEPETQDTTYLW |
| | | WVNNQSLPVSPRLELSSDNRTLTVFNIPRNDTTSYKCETQNPVS |
| | | VRRSDPVTLNVLYGPDAPTISPLNTPYRAGENLNLTCHAASNPT |
| | | AQYFWFVNGTFQQSTQELFIPNITVNNSGSYMCQAHNSATGLN |
| | | RTTVTAITVYAELPKPYITSNNSNPIEDKDAVTLTCEPETQDTTY |
| | | LWWVNNQSLSVSSRLELSNDNRTLTVFNIPRNDTTFYECETQNP |
| | | VSVRRSDPVTLNVLYGPDAPTISPLNTPYRAGENLNLSCHAASN |
| | | PAAQYSWFVNGTFQQSTQELFIPNITVNNSGSYMCQAHNSATG |
| | | LNRTTVTAITVYVELPKPYISSNNSNPIEDKDAVTLTCEPVAENT |
| | | TYLWWVNNQSLSVSPRLQLSNGNRILTLLSVTRNDTGPYECGI |
| | | QNSESAKRSDPVTLNVTYGPDTPIISPPDLSYRSGANLNLSCHS |
| | | DSNPSPQYSWLINGTLRQHTQVLFISKITSNNSGAYACFVSNLAT |
| | | GRNNSIVKNISVSSGDSAPGSSGLSARAHHHHHH |
| SEQ ID | 1-320 of | MGPPSAPPCRLHVPWKEVLLTASLLTFWNPPTTAKLTIESTPFNV |
| NO: 57 | CEACAM | AEGKEVLLLAHNLPQNRIGYSWYKGERVDGNSLIVGYVIGTQQ |
| | 6 | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPSKANYRPGENLNLSCHAAS |
| | | NPPAQYSWFINGTFQQSTQELFIPNITVNNSGSYMCQAHNSATG |
| | | LNRTTVTMITVSGHHHHHH |
| SEQ ID | 1-78 of | MESPSAPPHRWCIPWQRLLLTASLLTFWNPPTTAKLTIESTPFNV |
| NO: 58 | huCEA | AEGKEVLLLVHNLPQHLFGYSWYKGERVDGNRQIIGYVIGTQQ |
| | | ATPGPAYSGREIIYPNASLLIQNIIQN |
| SEQ ID | 398-687 of | ELSVDHSDPVILNVLYGPDDPTISPSYTYYRPGVNLSLSCHAAS |
| NO: 59 | huCEA | NPPAQYSWLIDGNIQQHTQELFISNITEKNSGLYTCQANNSASG |
| | | HSRTTVKTITVSAELPKPSISSNNSKPVEDKDAVAFTCEPEAQNT |
| | | TYLWWVNGQSLPVSPRLQLSNGNRTLTLFNVTRNDARAYVCGI |
| | | QNSVSANRSDPVTLDVLYGPDTPIISPPDSSYLSGANLNLSCHSA |
| | | SNPSPQYSWRINGIPQQHTQVLFIAKITPNNNGTYACFVSNLAT |
| | | GRNNSIVKSITVSASGTSPGLSAGA |
| SEQ ID | CEA-v | MESPSAPPHRWCIPWQRLLLTASLLTFWNPPTTAKLTIESTPFNV |
| NO: 60 | | AEGKEVLLLVHNLPQHLFGYSWYKGERVDGNRQIIGYVIGTQQ |
| | | ATPGPAYSGREIIYPNASLLIQNIIQNELSVDHSDPVILNVLYGPD |
| | | DPTISPSYTYYRPGVNLSLSCHAASNPPAQYSWLIDGNIQQHTQ |
| | | ELFISNITEKNSGLYTCQANNSASGHSRTTVKTITVSAELPKPSIS |
| | | SNNSKPVEDKDAVAFTCEPEAQNTTYLWWVNGQSLPVSPRLQL |
| | | SNGNRTLTLFNVTRNDARAYVCGIQNSVSANRSDPVTLDVLYG |
| | | PDTPIISPPDSSYLSGANLNLSCHSASNPSPQYSWRINGIPQQHT |
| | | QVLFIAKITPNNNGTYACFVSNLATGRNNSIVKSITVSASGTSPG |
| | | LSAGAHHHHHH |
| SEQ ID | 1-273 of | MGPPSAPPCRLHVPWKEVLLTASLLTFWNPPTTAKLTIESTPFNV |
| NO: 61 | CEACAM | AEGKEVLLLAHNLPQNRIGYSWYKGERVDGNSLIVGYVIGTQQ |
| | 6 | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTIS |
| SEQ ID | 596-687 of | PIISPPDSSYLSGANLNLSCHSASNPSPQYSWRINGIPQQHTQVL |
| NO: 62 | huCEA | FIAKITPNNNGTYACFVSNLATGRNNSIVKSITVSASGTSPGLSA |
| | | GA |
| SEQ ID | CHIM | MGPPSAPPCRLHVPWKEVLLTASLLTFWNPPTTAKLTIESTPFNV |
| NO: 63 | | AEGKEVLLLAHNLPQNRIGYSWYKGERVDGNSLIVGYVIGTQQ |
| | | ATPGPAYSGRETIYPNASLLIQNVTQNDTGFYTLQVIKSDLVNEE |
| | | ATGQFHVYPELPKPSISSNNSNPVEDKDAVAFTCEPEVQNTTYL |
| | | WWVNGQSLPVSPRLQLSNGNMTLTLLSVKRNDAGSYECEIQN |
| | | PASANRSDPVTLNVLYGPDVPTISPIISPPDSSYLSGANLNLSCHS |
| | | ASNPSPQYSWRINGIPQQHTQVLFIAKITPNNNGTYACFVSNLA |
| | | TGRNNSIVKSITVSASGTSPGLSAGAHHHHHH |
| SEQ ID | CEACAM | MGHLSAPLHRVRVPWQGLLLTASLLTFWNPPTTA QLTTESMPF |
| NO: 64 | 1 | NVAEGKEVLLLVHNLPQQLFGYSWYKGERVDGNRQIVGYAIG |
| | | TQQATPGPANSGRETIYPNASLLIQNVTQNDTGFYTLQVIKSDL |
| | | VNEEATGQFHVYPELPKPSISSNNSNPVEDKDAVAFTCEPETQD |
| | | TTYLWWINNQSLPVSPRLQLSNGNRTLTLLSVTRNDTGPYECEI |
| | | QNPVSANRSDPVTLNVTYGPDTPTISPSDTYYRPGANLSLSCYA |
| | | ASNPPAQYSWLINGTFQQSTQELFIPNITVNNSGSYTCHANNSV |
| | | TGCNRTTVKTIIVTELSPVVAKPQIKASKTTVTGDKDSVNLTCST |
| | | NDTGISIRWFFKNQSLPSSERMKLSQGNTTLSINPVKREDAGTY |
| | | WCEVFNPISKNQSDPIMLNVNYNALPQENGLSPGAIAGIVIGVV |
| | | ALVALIAVALACFLHFGKTGSSGPLQ |
| SEQ ID | CEACAM | MGPPSASPHRECIPWQGLLLTASLLNFWNPPTTAKLTIESMPLSV |
| NO: 65 | 3 | AEGKEVLLLVHNLPQHLFGYSWYKGERVDGNSLIVGYVIGTQ |
| | | QATPGAAYSGRETIYTNASLLIQNVTQNDIGFYTLQVIKSDLVN |
| | | EEATGQFHVYQENAPGLPVGAVAGIVTGVLVGVALVAALVCFL |
| | | LLAKTGRTSIQRDLKEQQPQALAPGRGPSHSSAFSMSPLSTAQA |
| | | PLPNPRTAASIYEELLKHDTNIYCRMDHKAEVAS |
| SEQ ID | CEACAM | MGSPSACPYRVCIPWQGLLLTASLLTFWNLPNSAQTNIDVVPFN |
| NO: 66 | 7 | VAEGKEVLLVVHNESQNLYGYNWYKGERVHANYRIIGYVKNI |
| | | SQENAPGPAHNGRETIYPNGTLLIQNVTHNDAGIYTLHVIKENL |
| | | VNEEVTRQFYVFSEPPKPSITSNNFNPVENKDIVVLTCQPETQNT |
| | | TYLWWVNNQSLLVSPRLLLSTDNRTLVLLSATKNDIGPYECEIQ |
| | | NPVGASRSDPVTLNVRYESVQASSPDLSAGTAVSIMIGVLAGM |
| | | ALI |
| SEQ ID | CEACAM | MGPISAPSCRWRIPWQGLLLTASLFTFWNPPTTAQLTIEAVPSNA |
| NO: 67 | 8 | AEGKEVLLLVHNLPQDPRGYNWYKGETVDANRRIIGYVISNQQ |
| | | ITPGPAYSNRETIYPNASLLMRNVTRNDTGSYTLQVIKLNLMSE |
| | | EVTGQFSVHPETPKPSISSNNSNPVEDKDAVAFTCEPETQNTTYL |
| | | WWVNGQSLPVSPRLQLSNGNRTLTLLSVTRNDVGPYECEIQNP |
| | | ASANFSDPVTLNVLYGPDAPTISPSDTYYHAGVNLNLSCHAAS |
| | | NPPSQYSWSVNGTFQQYTQKLFIPNITTKNSGSYACHTTNSATG |
| | | RNRTTVRMITVSDALVQGSSPGLSARATVSIMIGVLARVALI |
| SEQ ID | full-length | ATGGAGTCTCCCTCGGCCCCTCCCCACAGATGGTGCATCCCC |
| NO: 68 | human | TGGCAGAGGCTCCTGCTCACAGCCTCACTTCTAACCTTCTGG |
| | CEA DNA | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGACACAGGATTCTACACCCTACACGTCATAAAG |
| | | TCAGATCTTGTGAATGAAGAAGCAACTGGCCAGTTCCGGGT |
| | | ATACCCGGAGCTGCCCAAGCCCTCCATCTCCAGCAACAACTC |
| | | CAAACCCGTGGAGGACAAGGATGCTGTGGCCTTCACCTGTG |
| | | AACCTGAGACTCAGGACGCAACCTACCTGTGGTGGGTAAAC |
| | | AATCAGAGCCTCCCGGTCAGTCCCAGGCTGCAGCTGTCCAAT |
| | | GGCAACAGGACCCTCACTCTATTCAATGTCACAAGAAATGAC |
| | | ACAGCAAGCTACAAATGTGAAACCCAGAACCCAGTGAGTGC |
| | | CAGGCGCAGTGATTCAGTCATCCTGAATGTCCTCTATGGCCC |
| | | GGATGCCCCCACCATTTCCCCTCTAAACACATCTTACAGATCA |
| | | GGGGAAAATCTGAACCTCTCCTGCCACGCAGCCTCTAACCC |
| | | ACCTGCACAGTACTCTTGGTTTGTCAATGGGACTTTCCAGCA |
| | | ATCCACCCAAGAGCTCTTTATCCCCAACATCACTGTGAATAAT |
| | | AGTGGATCCTATACGTGCCAAGCCCATAACTCAGACACTGGC |
| | | CTCAATAGGACCACAGTCACGACGATCACAGTCTATGCAGAG |
| | | CCACCCAAACCCTTCATCACCAGCAACAACTCCAACCCCGT |
| | | GGAGGATGAGGATGCTGTAGCCTTAACCTGTGAACCTGAGAT |
| | | TCAGAACACAACCTACCTGTGGTGGGTAAATAATCAGAGCCT |
| | | CCCGGTCAGTCCCAGGCTGCAGCTGTCCAATGACAACAGGA |
| | | CCCTCACTCTACTCAGTGTCACAAGGAATGATGTAGGACCCT |
| | | ATGAGTGTGGAATCCAGAACGAATTAAGTGTTGACCACAGC |
| | | GACCCAGTCATCCTGAATGTCCTCTATGGCCCAGACGACCCC |
| | | ACCATTTCCCCCTCATACACCTATTACCGTCCAGGGGTGAACC |
| | | TCAGCCTCTCCTGCCATGCAGCCTCTAACCCACCTGCACAGT |
| | | ATTCTTGGCTGATTGATGGGAACATCCAGCAACACACACAAG |
| | | AGCTCTTTATCTCCAACATCACTGAGAAGAACAGCGGACTCT |
| | | ATACCTGCCAGGCCAATAACTCAGCCAGTGGCCACAGCAGG |
| | | ACTACAGTCAAGACAATCACAGTCTCTGCGGAGCTGCCCAA |
| | | GCCCTCCATCTCCAGCAACAACTCCAAACCCGTGGAGGACA |
| | | AGGATGCTGTGGCCTTCACCTGTGAACCTGAGGCTCAGAAC |
| | | ACAACCTACCTGTGGTGGGTAAATGGTCAGAGCCTCCCAGTC |
| | | AGTCCCAGGCTGCAGCTGTCCAATGGCAACAGGACCCTCAC |
| | | TCTATTCAATGTCACAAGAAATGACGCAAGAGCCTATGTATG |
| | | TGGAATCCAGAACTCAGTGAGTGCAAACCGCAGTGACCCAG |
| | | TCACCCTGGATGTCCTCTATGGGCCGGACACCCCCATCATTTC |
| | | CCCCCCAGACTCGTCTTACCTTTCGGGAGCGAACCTCAACCT |
| | | CTCCTGCCACTCGGCCTCTAACCCATCCCCGCAGTATTCTTGG |
| | | CGTATCAATGGGATACCGCAGCAACACACACAAGTTCTCTTT |
| | | ATCGCCAAAATCACGCCAAATAATAACGGGACCTATGCCTGT |
| | | TTTGTCTCTAACTTGGCTACTGGCCGCAATAATTCCATAGTCA |
| | | AGAGCATCACAGTCTCTGCATCTGGAACTTCTCCTGGTCTCT |
| | | CAGCTGGGGCCACTGTCGGCATCATGATTGGAGTGCTGGTTG |
| | | GGGTTGCTCTGATATAG |
| SEQ ID | monkey | ATGGGGTCTCCCTCAGCCCCTCTTCACAGATGGTGCATCCCC |
| NO: 69 | CEA DNA | TGGCAGACGCTCCTGCTCACAGCCTCACTTCTAACCTTCTGG |
| | | AACCCGCCCACCACTGCCCAGCTCACTATTGAATCCAGGCCG |
| | | TTCAATGTTGCAGAGGGGAAGGAGGTTCTTCTACTTGCCCAC |
| | | AATGTGTCCCAGAATCTTTTTGGCTACATTTGGTACAAGGGA |
| | | GAAAGAGTGGATGCCAGCCGTCGAATTGGATCATGTGTAATA |
| | | AGAACTCAACAAATTACCCCAGGGCCCGCACACAGCGGTCG |
| | | AGAGACAATAGACTTCAATGCATCCCTGCTGATCCACAATGT |
| | | CACCCAGAGTGACACAGGATCCTACACCATACAAGTCATAAA |
| | | GGAAGATCTTGTGAATGAAGAAGCAACTGGCCAGTTCCGGG |
| | | TATACCCGGAGCTGCCCAAGCCCTACATCTCCAGCAACAACT |
| | | CCAACCCCGTGGAGGACAAGGATGCTGTGGCCTTAACCTGT |
| | | GAACCTGAGACTCAGGACACAACCTACCTGTGGTGGGTAAA |
| | | CAATCAGAGCCTCCCGGTCAGTCCCAGGCTGGAGCTGTCCA |
| | | GTGACAACAGGACCCTCACTGTATTCAATATTCCAAGAAATG |
| | | ACACAACATCCTACAAATGTGAAACCCAGAACCCAGTGAGT |
| | | GTCAGACGCAGCGACCCAGTCACCCTGAACGTCCTCTATGG |
| | | CCCGGATGCGCCCACCATTTCCCCTCTAAACACACCTTACAG |
| | | AGCAGGGGAAAATCTGAACCTCACCTGCCACGCAGCCTCTA |
| | | ACCCAACTGCACAGTACTTTTGGTTTGTCAATGGGACGTTCC |
| | | AGCAATCCACACAAGAGCTCTTTATACCCAACATCACCGTGA |
| | | ATAATAGCGGATCCTATATGTGCCAAGCCCATAACTCAGCCAC |
| | | TGGCCTCAATAGGACCACAGTCACGGCGATCACAGTCTACGC |
| | | GGAGCTGCCCAAGCCCTACATCACCAGCAACAACTCCAACC |
| | | CCATAGAGGACAAGGATGCTGTGACCTTAACCTGTGAACCTG |
| | | AGACTCAGGACACAACCTACCTGTGGTGGGTAAACAATCAG |
| | | AGCCTCTCGGTCAGTTCCAGGCTGGAGCTGTCCAATGACAA |
| | | CAGGACCCTCACTGTATTCAATATTCCAAGAAACGACACAAC |
| | | GTTCTACGAATGTGAGACCCAGAACCCAGTGAGTGTCAGAC |
| | | GCAGCGACCCAGTCACCCTGAATGTCCTCTATGGCCCGGATG |
| | | CGCCCACCATTTCCCCTCTAAACACACCTTACAGAGCAGGGG |
| | | AAAATCTGAACCTCTCCTGCCACGCAGCCTCTAACCCAGCTG |
| | | CACAGTACTCTTGGTTTGTCAATGGGACGTTCCAGCAATCCA |
| | | CACAAGAGCTCTTTATACCCAACATCACCGTGAATAATAGCG |
| | | GATCCTATATGTGCCAAGCCCATAACTCAGCCACTGGCCTCA |
| | | ATAGGACCACAGTCACGGCGATCACAGTCTATGTGGAGCTGC |
| | | CCAAGCCCTACATCTCCAGCAACAACTCCAACCCCATAGAGG |
| | | ACAAGGATGCTGTGACCTTAACCTGTGAACCTGTGGCTGAG |
| | | AACACAACCTACCTGTGGTGGGTAAACAATCAGAGCCTCTC |
| | | GGTCAGTCCCAGGCTGCAGCTCTCCAATGGCAACAGGATCCT |
| | | CACTCTACTCAGTGTCACACGGAATGACACAGGACCCTATGA |
| | | ATGTGGAATCCAGAACTCAGAGAGTGCAAAACGCAGTGACC |
| | | CAGTCACCCTGAATGTCACCTATGGCCCAGACACCCCCATCA |
| | | TATCCCCCCCAGACTTGTCTTACCGTTCGGGAGCAAACCTCA |
| | | ACCTCTCCTGCCACTCGGACTCTAACCCATCCCCGCAGTATTC |
| | | TTGGCTTATCAATGGGACACTGCGGCAACACACACAAGTTCT |
| | | CTTTATCTCCAAAATCACATCAAACAATAGCGGGGCCTATGCC |
| | | TGTTTTGTCTCTAACTTGGCTACCGGTCGCAATAACTCCATAG |
| | | TCAAGAACATCTCAGTCTCCTCTGGCGATTCAGCACCTGGAA |
| | | GTTCTGGTCTCTCAGCTAGGGCTACTGTCGGCATCATAATTGG |
| | | AATGCTGGTTGGGGTTGCTCTGATGTAG |
| SEQ ID | full-length | ATGGGACCCCCCTCAGCCCCTCCCTGCAGATTGCATGTCCCC |
| NO: 70 | human | TGGAAGGAGGTCCTGCTCACAGCCTCACTTCTAACCTTCTGG |
| | CEACAM | AACCCACCCACCACTGCCAAGCTCACTATTGAATCCACGCCG |
| | 6 DNA | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCTCAAAGGCCAATTACCGTC |
| | | CAGGGGAAAATCTGAACCTCTCCTGCCACGCAGCCTCTAAC |
| | | CCACCTGCACAGTACTCTTGGTTTATCAATGGGACGTTCCAG |
| | | CAATCCACACAAGAGCTCTTTATCCCCAACATCACTGTGAAT |
| | | AATAGCGGATCCTATATGTGCCAAGCCCATAACTCAGCCACTG |
| | | GCCTCAATAGGACCACAGTCACGATGATCACAGTCTCTGGAA |
| | | GTGCTCCTGTCCTCTCAGCTGTGGCCACCGTCGGCATCACGA |
| | | TTGGAGTGCTGGCCAGGGTGGCTCTGATATAG |
| SEQ ID | 1-687 of | ATGGAGTCTCCCTCGGCCCCTCCCCACAGATGGTGCATCCCC |
| NO: 71 | huCEA | TGGCAGAGGCTCCTGCTCACAGCCTCACTTCTAACCTTCTGG |
| | DNA | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGACACAGGATTCTACACCCTACACGTCATAAAG |
| | | TCAGATCTTGTGAATGAAGAAGCAACTGGCCAGTTCCGGGT |
| | | ATACCCGGAGCTGCCCAAGCCCTCCATCTCCAGCAACAACTC |
| | | CAAACCCGTGGAGGACAAGGATGCTGTGGCCTTCACCTGTG |
| | | AACCTGAGACTCAGGACGCAACCTACCTGTGGTGGGTAAAC |
| | | AATCAGAGCCTCCCGGTCAGTCCCAGGCTGCAGCTGTCCAAT |
| | | GGCAACAGGACCCTCACTCTATTCAATGTCACAAGAAATGAC |
| | | ACAGCAAGCTACAAATGTGAAACCCAGAACCCAGTGAGTGC |
| | | CAGGCGCAGTGATTCAGTCATCCTGAATGTCCTCTATGGCCC |
| | | GGATGCCCCCACCATTTCCCCTCTAAACACATCTTACAGATCA |
| | | GGGGAAAATCTGAACCTCTCCTGCCACGCAGCCTCTAACCC |
| | | ACCTGCACAGTACTCTTGGTTTGTCAATGGGACTTTCCAGCA |
| | | ATCCACCCAAGAGCTCTTTATCCCCAACATCACTGTGAATAAT |
| | | AGTGGATCCTATACGTGCCAAGCCCATAACTCAGACACTGGC |
| | | CTCAATAGGACCACAGTCACGACGATCACAGTCTATGCAGAG |
| | | CCACCCAAACCCTTCATCACCAGCAACAACTCCAACCCCGT |
| | | GGAGGATGAGGATGCTGTAGCCTTAACCTGTGAACCTGAGAT |
| | | TCAGAACACAACCTACCTGTGGTGGGTAAATAATCAGAGCCT |
| | | CCCGGTCAGTCCCAGGCTGCAGCTGTCCAATGACAACAGGA |
| | | CCCTCACTCTACTCAGTGTCACAAGGAATGATGTAGGACCCT |
| | | ATGAGTGTGGAATCCAGAACGAATTAAGTGTTGACCACAGC |
| | | GACCCAGTCATCCTGAATGTCCTCTATGGCCCAGACGACCCC |
| | | ACCATTTCCCCCTCATACACCTATTACCGTCCAGGGGTGAACC |
| | | TCAGCCTCTCCTGCCATGCAGCCTCTAACCCACCTGCACAGT |
| | | ATTCTTGGCTGATTGATGGGAACATCCAGCAACACACACAAG |
| | | AGCTCTTTATCTCCAACATCACTGAGAAGAACAGCGGACTCT |
| | | ATACCTGCCAGGCCAATAACTCAGCCAGTGGCCACAGCAGG |
| | | ACTACAGTCAAGACAATCACAGTCTCTGCGGAGCTGCCCAA |
| | | GCCCTCCATCTCCAGCAACAACTCCAAACCCGTGGAGGACA |
| | | AGGATGCTGTGGCCTTCACCTGTGAACCTGAGGCTCAGAAC |
| | | ACAACCTACCTGTGGTGGGTAAATGGTCAGAGCCTCCCAGTC |
| | | AGTCCCAGGCTGCAGCTGTCCAATGGCAACAGGACCCTCAC |
| | | TCTATTCAATGTCACAAGAAATGACGCAAGAGCCTATGTATG |
| | | TGGAATCCAGAACTCAGTGAGTGCAAACCGCAGTGACCCAG |
| | | TCACCCTGGATGTCCTCTATGGGCCGGACACCCCCATCATTTC |
| | | CCCCCCAGACTCGTCTTACCTTTCGGGAGCGAACCTCAACCT |
| | | CTCCTGCCACTCGGCCTCTAACCCATCCCCGCAGTATTCTTGG |
| | | CGTATCAATGGGATACCGCAGCAACACACACAAGTTCTCTTT |
| | | ATCGCCAAAATCACGCCAAATAATAACGGGACCTATGCCTGT |
| | | TTTGTCTCTAACTTGGCTACTGGCCGCAATAATTCCATAGTCA |
| | | AGAGCATCACAGTCTCTGCATCTGGAACTTCTCCTGGTCTCT |
| | | CAGCTGGGGCCCATCACCATCACCATCAC |
| SEQ ID | 1-690 of | ATGGGGTCTCCCTCAGCCCCTCTTCACAGATGGTGCATCCCC |
| NO: 72 | monkey | TGGCAGACGCTCCTGCTCACAGCCTCACTTCTAACCTTCTGG |
| | CEA DNA | AACCCGCCCACCACTGCCCAGCTCACTATTGAATCCAGGCCG |
| | | TTCAATGTTGCAGAGGGGAAGGAGGTTCTTCTACTTGCCCAC |
| | | AATGTGTCCCAGAATCTTTTTGGCTACATTTGGTACAAGGGA |
| | | GAAAGAGTGGATGCCAGCCGTCGAATTGGATCATGTGTAATA |
| | | AGAACTCAACAAATTACCCCAGGGCCCGCACACAGCGGTCG |
| | | AGAGACAATAGACTTCAATGCATCCCTGCTGATCCACAATGT |
| | | CACCCAGAGTGACACAGGATCCTACACCATACAAGTCATAAA |
| | | GGAAGATCTTGTGAATGAAGAAGCAACTGGCCAGTTCCGGG |
| | | TATACCCGGAGCTGCCCAAGCCCTACATCTCCAGCAACAACT |
| | | CCAACCCCGTGGAGGACAAGGATGCTGTGGCCTTAACCTGT |
| | | GAACCTGAGACTCAGGACACAACCTACCTGTGGTGGGTAAA |
| | | CAATCAGAGCCTCCCGGTCAGTCCCAGGCTGGAGCTGTCCA |
| | | GTGACAACAGGACCCTCACTGTATTCAATATTCCAAGAAATG |
| | | ACACAACATCCTACAAATGTGAAACCCAGAACCCAGTGAGT |
| | | GTCAGACGCAGCGACCCAGTCACCCTGAACGTCCTCTATGG |
| | | CCCGGATGCGCCCACCATTTCCCCTCTAAACACACCTTACAG |
| | | AGCAGGGGAAAATCTGAACCTCACCTGCCACGCAGCCTCTA |
| | | ACCCAACTGCACAGTACTTTTGGTTTGTCAATGGGACGTTCC |
| | | AGCAATCCACACAAGAGCTCTTTATACCCAACATCACCGTGA |
| | | ATAATAGCGGATCCTATATGTGCCAAGCCCATAACTCAGCCAC |
| | | TGGCCTCAATAGGACCACAGTCACGGCGATCACAGTCTACGC |
| | | GGAGCTGCCCAAGCCCTACATCACCAGCAACAACTCCAACC |
| | | CCATAGAGGACAAGGATGCTGTGACCTTAACCTGTGAACCTG |
| | | AGACTCAGGACACAACCTACCTGTGGTGGGTAAACAATCAG |
| | | AGCCTCTCGGTCAGTTCCAGGCTGGAGCTGTCCAATGACAA |
| | | CAGGACCCTCACTGTATTCAATATTCCAAGAAACGACACAAC |
| | | GTTCTACGAATGTGAGACCCAGAACCCAGTGAGTGTCAGAC |
| | | GCAGCGACCCAGTCACCCTGAATGTCCTCTATGGCCCGGATG |
| | | CGCCCACCATTTCCCCTCTAAACACACCTTACAGAGCAGGGG |
| | | AAAATCTGAACCTCTCCTGCCACGCAGCCTCTAACCCAGCTG |
| | | CACAGTACTCTTGGTTTGTCAATGGGACGTTCCAGCAATCCA |
| | | CACAAGAGCTCTTTATACCCAACATCACCGTGAATAATAGCG |
| | | GATCCTATATGTGCCAAGCCCATAACTCAGCCACTGGCCTCA |
| | | ATAGGACCACAGTCACGGCGATCACAGTCTATGTGGAGCTGC |
| | | CCAAGCCCTACATCTCCAGCAACAACTCCAACCCCATAGAGG |
| | | ACAAGGATGCTGTGACCTTAACCTGTGAACCTGTGGCTGAG |
| | | AACACAACCTACCTGTGGTGGGTAAACAATCAGAGCCTCTC |
| | | GGTCAGTCCCAGGCTGCAGCTCTCCAATGGCAACAGGATCCT |
| | | CACTCTACTCAGTGTCACACGGAATGACACAGGACCCTATGA |
| | | ATGTGGAATCCAGAACTCAGAGAGTGCAAAACGCAGTGACC |
| | | CAGTCACCCTGAATGTCACCTATGGCCCAGACACCCCCATCA |
| | | TATCCCCCCCAGACTTGTCTTACCGTTCGGGAGCAAACCTCA |
| | | ACCTCTCCTGCCACTCGGACTCTAACCCATCCCCGCAGTATTC |
| | | TTGGCTTATCAATGGGACACTGCGGCAACACACACAAGTTCT |
| | | CTTTATCTCCAAAATCACATCAAACAATAGCGGGGCCTATGCC |
| | | TGTTTTGTCTCTAACTTGGCTACCGGTCGCAATAACTCCATAG |
| | | TCAAGAACATCTCAGTCTCCTCTGGCGATTCAGCACCTGGAA |
| | | GTTCTGGTCTCTCAGCTAGGGCTCATCACCATCACCATCAC |
| SEQ ID | 1-320 of | ATGGGACCCCCCTCAGCCCCTCCCTGCAGATTGCATGTCCCC |
| NO: 73 | CEACAM | TGGAAGGAGGTCCTGCTCACAGCCTCACTTCTAACCTTCTGG |
| | 6 DNA | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCTCAAAGGCCAATTACCGTC |
| | | CAGGGGAAAATCTGAACCTCTCCTGCCACGCAGCCTCTAAC |
| | | CCACCTGCACAGTACTCTTGGTTTATCAATGGGACGTTCCAG |
| | | CAATCCACACAAGAGCTCTTTATCCCCAACATCACTGTGAAT |
| | | AATAGCGGATCCTATATGTGCCAAGCCCATAACTCAGCCACTG |
| | | GCCTCAATAGGACCACAGTCACGATGATCACAGTCTCTGGAC |
| | | ATCACCATCACCATCAC |
| SEQ ID | 1-78 of | ATGGAGTCTCCCTCGGCCCCTCCCCACAGATGGTGCATCCCC |
| NO: 74 | huCEA | TGGCAGAGGCTCCTGCTCACAGCCTCACTTCTAACCTTCTGG |
| | DNA | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAAT |
| SEQ ID | 398-687 of | GAATTAAGTGTTGACCACAGCGACCCAGTCATCCTGAATGTC |
| NO: 75 | huC3A | CTCTATGGCCCAGACGACCCCACCATTTCCCCCTCATACACCT |
| | DNA | ATTACCGTCCAGGGGTGAACCTCAGCCTCTCCTGCCATGCAG |
| | | CCTCTAACCCACCTGCACAGTATTCTTGGCTGATTGATGGGA |
| | | ACATCCAGCAACACACACAAGAGCTCTTTATCTCCAACATCA |
| | | CTGAGAAGAACAGCGGACTCTATACCTGCCAGGCCAATAACT |
| | | CAGCCAGTGGCCACAGCAGGACTACAGTCAAGACAATCACA |
| | | GTCTCTGCGGAGCTGCCCAAGCCCTCCATCTCCAGCAACAAC |
| | | TCCAAACCCGTGGAGGACAAGGATGCTGTGGCCTTCACCTG |
| | | TGAACCTGAGGCTCAGAACACAACCTACCTGTGGTGGGTAA |
| | | ATGGTCAGAGCCTCCCAGTCAGTCCCAGGCTGCAGCTGTCC |
| | | AATGGCAACAGGACCCTCACTCTATTCAATGTCACAAGAAAT |
| | | GACGCAAGAGCCTATGTATGTGGAATCCAGAACTCAGTGAGT |
| | | GCAAACCGCAGTGACCCAGTCACCCTGGATGTCCTCTATGGG |
| | | CCGGACACCCCCATCATTTCCCCCCCAGACTCGTCTTACCTTT |
| | | CGGGAGCGAACCTCAACCTCTCCTGCCACTCGGCCTCTAACC |
| | | CATCCCCGCAGTATTCTTGGCGTATCAATGGGATACCGCAGCA |
| | | ACACACACAAGTTCTCTTTATCGCCAAAATCACGCCAAATAA |
| | | TAACGGGACCTATGCCTGTTTTGTCTCTAACTTGGCTACTGGC |
| | | CGCAATAATTCCATAGTCAAGAGCATCACAGTCTCTGCATCTG |
| | | GAACTTCTCCTGGTCTCTCAGCTGGGGCC |
| SEQ ID | CEA-v | ATGGAGTCTCCCTCGGCCCCTCCCCACAGATGGTGCATCCCC |
| NO: 76 | DNA | TGGCAGAGGCTCCTGCTCACAGCCTCACTTCTAACCTTCTGG |
| | | AACCCGCCCACCACTGCCAAGCTCACTATTGAATCCACGCCG |
| | | TTCAATGTCGCAGAGGGGAAGGAGGTGCTTCTACTTGTCCAC |
| | | AATCTGCCCCAGCATCTTTTTGGCTACAGCTGGTACAAAGGT |
| | | GAAAGAGTGGATGGCAACCGTCAAATTATAGGATATGTAATA |
| | | GGAACTCAACAAGCTACCCCAGGGCCCGCATACAGTGGTCG |
| | | AGAGATAATATACCCCAATGCATCCCTGCTGATCCAGAACATC |
| | | ATCCAGAATGAATTAAGTGTTGACCACAGCGACCCAGTCATC |
| | | CTGAATGTCCTCTATGGCCCAGACGACCCCACCATTTCCCCC |
| | | TCATACACCTATTACCGTCCAGGGGTGAACCTCAGCCTCTCC |
| | | TGCCATGCAGCCTCTAACCCACCTGCACAGTATTCTTGGCTG |
| | | ATTGATGGGAACATCCAGCAACACACACAAGAGCTCTTTATC |
| | | TCCAACATCACTGAGAAGAACAGCGGACTCTATACCTGCCAG |
| | | GCCAATAACTCAGCCAGTGGCCACAGCAGGACTACAGTCAA |
| | | GACAATCACAGTCTCTGCGGAGCTGCCCAAGCCCTCCATCTC |
| | | CAGCAACAACTCCAAACCCGTGGAGGACAAGGATGCTGTGG |
| | | CCTTCACCTGTGAACCTGAGGCTCAGAACACAACCTACCTGT |
| | | GGTGGGTAAATGGTCAGAGCCTCCCAGTCAGTCCCAGGCTG |
| | | CAGCTGTCCAATGGCAACAGGACCCTCACTCTATTCAATGTC |
| | | ACAAGAAATGACGCAAGAGCCTATGTATGTGGAATCCAGAA |
| | | CTCAGTGAGTGCAAACCGCAGTGACCCAGTCACCCTGGATG |
| | | TCCTCTATGGGCCGGACACCCCCATCATTTCCCCCCCAGACT |
| | | CGTCTTACCTTTCGGGAGCGAACCTCAACCTCTCCTGCCACT |
| | | CGGCCTCTAACCCATCCCCGCAGTATTCTTGGCGTATCAATGG |
| | | GATACCGCAGCAACACACACAAGTTCTCTTTATCGCCAAAAT |
| | | CACGCCAAATAATAACGGGACCTATGCCTGTTTTGTCTCTAAC |
| | | TTGGCTACTGGCCGCAATAATTCCATAGTCAAGAGCATCACA |
| | | GTCTCTGCATCTGGAACTTCTCCTGGTCTCTCAGCTGGGGCC |
| | | CATCACCATCACCATCAC |
| SEQ ID | 1-273 of | ATGGGACCCCCCTCAGCCCCTCCCTGCAGATTGCATGTCCCC |
| NO: 77 | CEACAM | TGGAAGGAGGTCCTGCTCACAGCCTCACTTCTAACCTTCTGG |
| | 6 DNA | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCC |
| SEQ ID | 596-687 of | CCCATCATTTCCCCCCCAGACTCGTCTTACCTTTCGGGAGCG |
| NO: 78 | huCEA | AACCTCAACCTCTCCTGCCACTCGGCCTCTAACCCATCCCCG |
| | DNA | CAGTATTCTTGGCGTATCAATGGGATACCGCAGCAACACACA |
| | | CAAGTTCTCTTTATCGCCAAAATCACGCCAAATAATAACGGG |
| | | ACCTATGCCTGTTTTGTCTCTAACTTGGCTACTGGCCGCAATA |
| | | ATTCCATAGTCAAGAGCATCACAGTCTCTGCATCTGGAACTT |
| | | CTCCTGGTCTCTCAGCTGGGGCC |
| SEQ ID | CHIM | ATGGGACCCCCCTCAGCCCCTCCCTGCAGATTGCATGTCCCC |
| NO: 79 | DNA | TGGAAGGAGGTCCTGCTCACAGCCTCACTTCTAACCTTCTGG |
| | | AACCCACCCACCACTGCCAAGCTCACTATTGAATCCACGCCG |
| | | TTCAATGTCGCAGAGGGGAAGGAGGTTCTTCTACTCGCCCAC |
| | | AACCTGCCCCAGAATCGTATTGGTTACAGCTGGTACAAAGGC |
| | | GAAAGAGTGGATGGCAACAGTCTAATTGTAGGATATGTAATA |
| | | GGAACTCAACAAGCTACCCCAGGGCCCGCATACAGTGGTCG |
| | | AGAGACAATATACCCCAATGCATCCCTGCTGATCCAGAACGT |
| | | CACCCAGAATGACACAGGATTCTATACCCTACAAGTCATAAA |
| | | GTCAGATCTTGTGAATGAAGAAGCAACCGGACAGTTCCATGT |
| | | ATACCCGGAGCTGCCCAAGCCCTCCATCTCCAGCAACAACTC |
| | | CAACCCCGTGGAGGACAAGGATGCTGTGGCCTTCACCTGTG |
| | | AACCTGAGGTTCAGAACACAACCTACCTGTGGTGGGTAAAT |
| | | GGTCAGAGCCTCCCGGTCAGTCCCAGGCTGCAGCTGTCCAA |
| | | TGGCAACATGACCCTCACTCTACTCAGCGTCAAAAGGAACG |
| | | ATGCAGGATCCTATGAATGTGAAATACAGAACCCAGCGAGTG |
| | | CCAACCGCAGTGACCCAGTCACCCTGAATGTCCTCTATGGCC |
| | | CAGATGTCCCCACCATTTCCCCCATCATTTCCCCCCCAGACTC |
| | | GTCTTACCTTTCGGGAGCGAACCTCAACCTCTCCTGCCACTC |
| | | GGCCTCTAACCCATCCCCGCAGTATTCTTGGCGTATCAATGGG |
| | | ATACCGCAGCAACACACACAAGTTCTCTTTATCGCCAAAATC |
| | | ACGCCAAATAATAACGGGACCTATGCCTGTTTTGTCTCTAACT |
| | | TGGCTACTGGCCGCAATAATTCCATAGTCAAGAGCATCACAG |
| | | TCTCTGCATCTGGAACTTCTCCTGGTCTCTCAGCTGGGGCCC |
| | | ATCACCATCACCATCAC |

### Example 2. Cloning and Sequence Analysis of CEA Antibodies

Murine hybridoma cells were harvested to prepare total RNAs using an Ultrapure RNA kit (Cat. No. 74104, QIAGEN, Germany) based on the manufacturer's protocol. The 1^{st} strand cDNAs were synthesized using a cDNA synthesis kit from Invitrogen (Cat. No. 18080-051) and PCR amplification of VH and VL genes of murine monoclonal antibodies was performed using a PCR kit (Cat. No. CW0686, CWBio, Beijing, China). The oligo primers used for antibody cDNAs cloning of heavy chain variable region (VH) and kappa light chain variable region (VL) were synthesized based on the sequences reported previously (Brocks et al., Mol Med. 2001 7(7):461-9). PCR products were then subcloned into the pEASY-Blunt cloning vector (Cat. No. CB101-02, TransGen, China) and sequenced. The amino acid sequences of V_{H} and VL regions were determined from the DNA sequencing results.

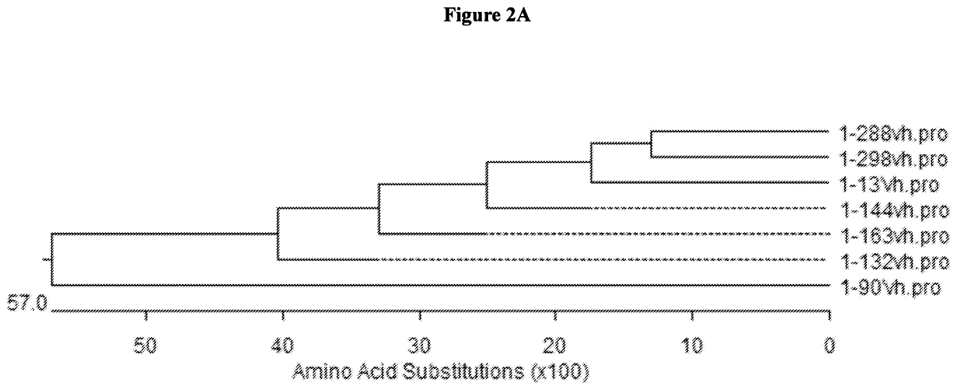

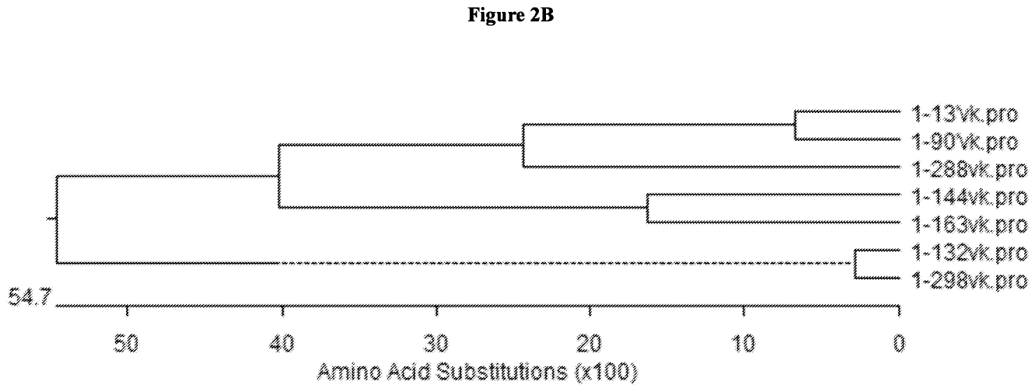

The monoclonal antibodies were analyzed by comparing sequence homology and grouped based on sequence similarity (FIG. 2). Complementary determinant regions (CDRs) were defined based on the IMGT (Lefranc et al., 1999 Nucleic Acids Research 27:209-212) system by sequence annotation. The amino acid sequences of a representative clone BGA13 are listed in Table 3.

TABLE 3
amino acid sequences

| | | | |
|---|---|---|---|
| BGA13 | SEQ ID | HCDR1 | GYIFTSYW |
| (IMGT) | NO: 80 | (IMGT) | |
| | SEQ ID | HCDR2 | INPNTGYT |
| | NO: 81 | (IMGT) | |
| | SEQ ID | HCDR3 | AREYGNYNYPLDY |
| | NO: 3 | (IMGT) | |
| | SEQ ID | LCDR1 | ENIYGY |
| | NO: 82 | (IMGT) | |
| | SEQ ID | LCDR2 | NA |
| | NO: 83 | (IMGT) | |
| | SEQ ID | LCDR3 | QHHYGTPYT |
| | NO: 84 | (IMGT) | |
| BGA13 | SEQ ID | VH | QVQLQQSGAELAKSGASVKMSCKASGYIFT |
| | | | SYWLHWVKQRPGQGLEWIGYINPNTGYTNY |
| | NO: 85 | | SQKFKDKATLTADKSSSTAYMQLSSLTSED |
| | | | SAVYYCAREYGNYNYPLDYWGQGTSVTVSS |
| | SEQ ID | VL | DIQMTQSPASLSASVGETVTITCRASENIY |
| | NO: 86 | | GYLAWYQQKQGKSPQLLVFNAKNLVEGVPS |
| | | | RFSGSGSGTQFSLKINSLQPEDFGSYYCQH |
| | | | HYGTPYTFGGGTKLEIK |

### Example 3. Binding Profiles Determination of Purified Murine Anti-CEA Antibodies

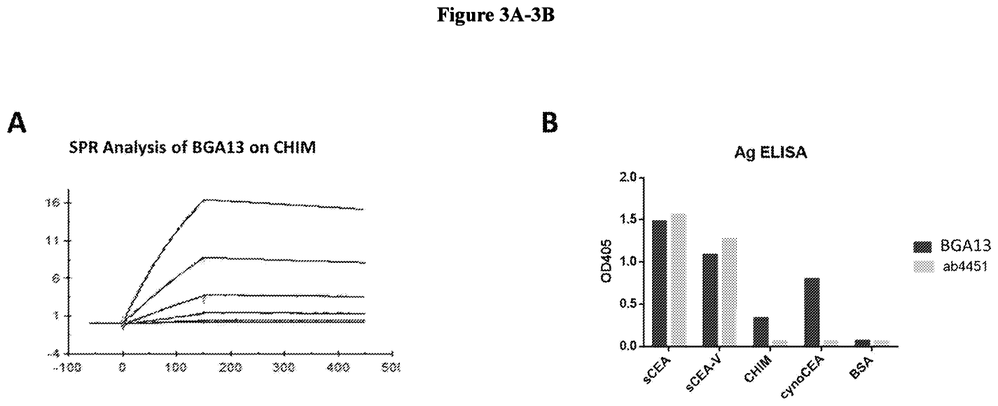

The CEA antibodies with specific binding for CEA as shown by ELISA and FACS, as well as without sCEA interference were characterized for their binding kinetics by SPR assays using BIAcore™ T-200 (GE Life Sciences) (FIG. 3A). Briefly, anti-murine IgG antibody was immobilized on an activated CM5 biosensor chip (Cat. No. BR100530, GE Life Sciences). Purified murine antibodies were flowed over the chip surface and captured by anti-murine IgG antibody. Then a serial dilution (6.0 nM to 2150 nM) of purified CHIM, CEA-v, CEA or monkey CEA recombinant proteins were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of BGA13 is shown below in Table 4.

The binding profiles of BGA13 were checked via antigen ELISA, the bindings of purified BGA13 to huCEA and monkey CEA were observed, these indicated BGA13 is a weak binder to soluble huCEA and monkey CEA, or soluble CEA has a different conformation when immobilized (FIG. 3B). For this experiment, sCEA, CHIM, monkey CEA, CEA-v and BSA were coated in 96-well plates at a high concentration of 10 μg/ml overnight at 4° C. BGA13 or a control antibody ab4451 (Cat. No. ab4451, abcam, USA) were incubated for 1 hour at a concentration of 2 μg/ml. The HRP-linked anti-mouse IgG antibody (Cat. No. 7076S, Cell Signaling Technology, USA) and substrate (Cat. No. 00-4201-56, eBioscience, USA) were used for development, and absorbance signal at the wavelength of 450 nm was measured using a plate reader (SpectraMax Paradigm, Molecular Devices, USA).

TABLE 4
Comparison of BGA13 binding affinities by SPR

| Antigen | KD (M) |
|---|---|
| sCEA | ND |
| sCEA-v | 1.50E−07 |
| CHIM | 1.20E−07 |
| Monkey | ND (not detectable |
| CEA | due to weak binding |

### Example 4. Effects of Recombinant Soluble CEA on Binding of BGA13 to CEA Expressing Cells

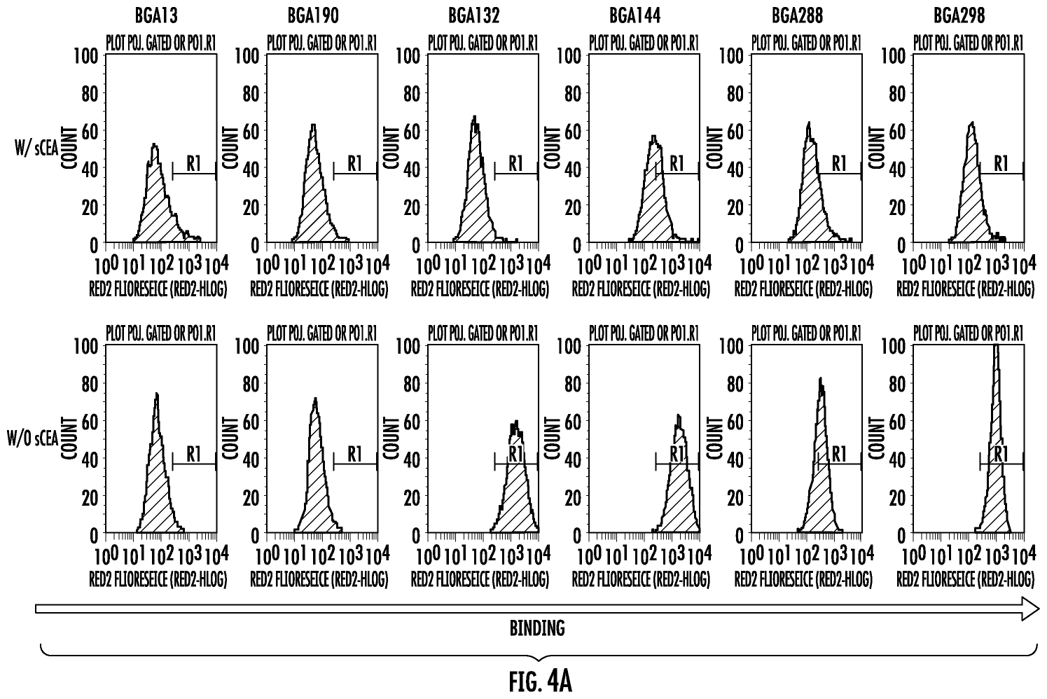

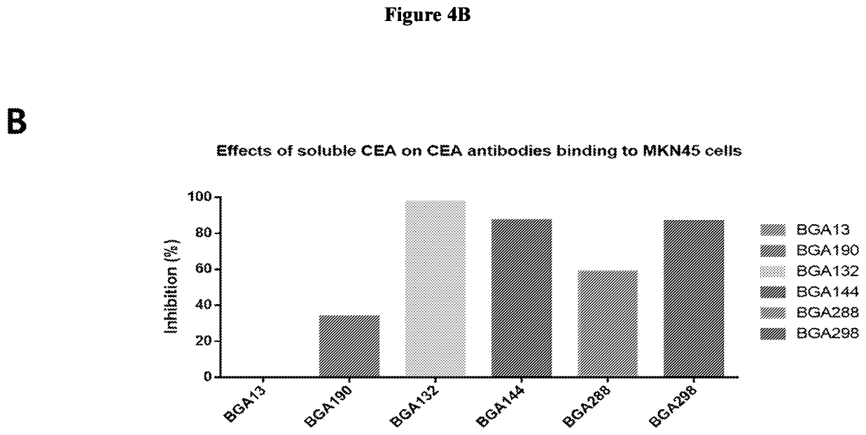

The presence of soluble CEA on the specific binding of various CEA antibodies to CEA expressing cells was evaluated via flow cytometry. In brief, human CEA-expressing cells (10⁵ cells/well) were incubated with 2 μg/ml purified CEA murine monoclonal antibodies in the presence of 20 μg/ml extra recombinant soluble CEA proteins, followed by binding with Alexa Fluro-647-labeled goat anti-mouse IgG antibody (Cat. No. A0473, Beyotime Biotechnology, China). Cell fluorescence was quantified using a flow cytometer (Guava easyCyte™ 8HT, Merck-Millipore, USA). As shown in FIGS. 4A and 4B, the binding of BGA13 to CEA expressing cells were not affected by the presence of soluble CEA.

### Example 5. Humanization of the Murine Anti-Human CEA Antibodies mAb Humanization and Engineering

For humanization of BGA13, human germline IgG genes were searched for sequences that share high degrees of homology with the cDNA sequences of BGA13 variable regions by sequence comparisons in the human immunoglobulin gene databases at IMGT and NCBI. The human IGVH and IGVL genes that are present in human antibody repertoires with high frequencies (Glanville et al., 2009 PNAS 106:20216-20221) and are highly homologous to BGA13 were selected as the templates for humanization. Before humanization, BGA13 heavy and light chain variable domains were fused to a wild type human IgG1 constant region designated as human IgG1 wt (SEQ ID NO:87) and a human kappa constant (CL) region (SEQ ID NO:88), respectively.

TABLE 5
amino acid sequences

| | | |
|---|---|---|
| SEQ ID | ASTKGPSVFPLAPSSKSTSGGTAALGCLVKDYFP | IgG1wt |
| NO: 87 | EPVTVSWNSGALTSGVHTFPAVLQSSGLYSLSSV | |
| | VTVPSSSLGTQTYICNVNHKPSNTKVDKKVEPKS | |
| | CDKTHTCPPCPAPELLGGPSVFLFPPKPKDTLMI | |
| | SRTPEVTCVVVDVSHEDPEVKFNWYVDGVEVHNA | |
| | KTKPREEQYNSTYRVVSVLTVLHQDWLNGKEYKC | |
| | KVSNKALPAPIEKTISKAKGQPREPQVYTLPPSR | |
| | DELTKNQVSLTCLVKGFYPSDIAVEWESNGQPEN | |
| | NYKTTPPVLDSDGSFFLYSKLTVDKSRWQQGNVF | |
| | SCSVMHEALHNHYTQKSLSLSPGK | |
| SEQ ID | RTVAAPSVFIFPPSDEQLKSGTASVVCLLNNFYP | CL |
| NO: 88 | REAKVQWKVDNALQSGNSQESVTEQDSKDSTYSL | |
| | SSTLTLSKADYEKHKVYACEVTHQGLSSPVTKSF | |
| | NRGEC | |

Humanization was carried out by CDR-grafting (Methods in Molecular Biology, Vol 248: Antibody Engineering, Methods and Protocols, Humana Press) and the BGA13 antibody was engineered in the human IgG1 format. In the initial round of humanization, mutations from murine to human amino acid residues in framework regions were guided by the simulated 3D structure, and the murine framework residues of structural importance for maintaining the canonical structures of CDRs were retained in the 1st version of humanized antibody BGA13, BGA131 (the amino acid sequences of the heavy chain and light chain are set forth in SEQ ID NOs: 89 and 90).

TABLE 6
amino acid sequences

| | | |
|---|---|---|
| SEQ ID | QVQLVQSGAEVKKPGASVKVSCKASGYIFTSYW | BGA131 |
| NO: 89 | LHWVRQAPGQGLEWIGYINPNTGYTNYSQKFQG | heavy |
| | RVTMTRDTSTSTVYMELSSLRSEDTAVYYCARE | chain |
| | YGNYNYPLDYWGQGTLVTVSSASTKGPSVFPLA | |
| | PSSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTC | |
| | VVVDVSHEDPEVKFNWYVDGVEVHNAKTKPREE | |
| | QYNSTYRVVSVLTVLHQDWLNGKEYKCKVSNKA | |
| | LPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VMHEALHNHYTQKSLSLSPGK | |
| SEQ ID | DIQMTQSPSSLSASVGDRVTITCRASENIYGYL | BGA131 |
| NO: 90 | AWYQQKPGKVPKLLIYNAKNLVEGVPSRFSGSG | light |
| | SGTDFTLTISSLQPEDVATYYCQHHYGTPYTFG | chain |
| | QGTKVEIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |

Specifically, CDRs of BGA13 VL were grafted into the frameworks of human germline variable gene IGVK1-27 with 2 murine framework residues (N66 and V68) retained (the amino acid sequence of the light chain variable domain is set forth in SEQ ID NO: 92). CDRs of BGA13 V_{H} were grafted into the frameworks of human germline variable gene IGVH1-46 with 5 murine framework (L39, 153, Y55, N66, S68) residues retained (the amino acid sequence of the heavy chain variable domain is set for in SEQ ID NO: 91).

TABLE 7
amino acid sequences

| | | |
|---|---|---|
| SEQ ID | QVQLVQSGAEVKKPGASVKVSCKASGYIFTSYW | BGA131 |
| NO: 91 | LHWVRQAPGQGLEWIGYINPNTGYTNYSQKFQG | VH |
| | RVTMTRDTSTSTVYMELSSLRSEDTAVYYCARE | |
| | YGNYNYPLDYWGQGTLVTVSS | |
| SEQ ID | DIQMTQSPSSLSASVGDRVTITCRASENIYGYL | BGA131 |
| NO: 92 | AWYQQKPGKVPKLLIYNAKNLVEGVPSRFSGSG | VL |
| | SGTDFTLTISSLQPEDVATYYCQHHYGTPYTFG | |
| | QGTKVEIK | |

BGA13-1 was constructed as human full-length antibody format using in-house developed expression vectors that contain constant regions of a wild type human IgG1 with easy adapting subcloning sites. Expression and preparation of BGA13-1 antibody was achieved by co-transfection of the above two constructs into 293G cells and by purification using a Protein A column (Cat. No. 17543802, GE Life Sciences). The purified antibodies were concentrated to 0.5-5 mg/mL in PBS and stored in aliquots in −80° C. freezer.

Using BGA131, an additional number of single or multiple amino acid changes were made, converting the human residues in framework regions of VH and VL to corresponding murine germline residues, which include V68A, R72A and V79A in VH and V43S in VL, respectively. This resulted BGA132 (V68A, R72A in VH), BGA133 (V79A in VH), BGA134 (V68A, R72A, V79A in VH), BGA135 (V43S in VL), BGA136 (V68A, R72A in VH, and V43S in VL), BGA137 (V79A in VH, V43S in VL) and BGA138 (V68A, R72A, V79A, in VH and V43S in VL). All antibodies which contained modifications had similar binding activities to BGA131, and none of the changes abolished binding.

In order to remove post-translational modification (PTM) sites, further engineering was made by introducing mutations in CDRs and framework regions based on the BGA131 sequence, which include N52T, N54Q, N59S, N102G, N104Q and S61A amino acid changes in the VH region. This resulted in BGA131A (N52T (VH)), BGA131B (N54Q (VH)), BGA131C (N59S (VH)), BGA131D (N102G (VH)), BGA131E (N104Q (VH)) and BGA131F (N54Q, N59S, S61A (VH)) and all of the antibodies had similar binding specificity to BGA131, with none of the changes abolishing binding. While maintaining specificity, amino acid compositions and expression levels were also considered. All humanization mutations were made using primers containing mutations at specific positions and a site directed mutagenesis kit (Cat. FM111-02, TransGen, Beijing, China). The desired mutations were verified by sequence analysis. Comparing to BGA13-1, BGA13-1F had significantly reduced binding affinities with no glycosylation sites but had a high expression level (Table 8).

TABLE 8
amino acid sequences

| | | |
|---|---|---|
| SEQ ID | DIQMTQSPSSLSASVGDRVTITCRASENIYGY | BGA13- |
| NO: 93 | LAWYQQKPGKVPKLLIYNAKNLVEGVPSRFSG | 1 VL |
| | SGSGTDFTLTISSLQPEDVATYYCQHHYGTPY | |
| | TFGQGTKVEIK | |
| SEQ ID | QVQLVQSGAEVKKPGASVKVSCKASGYIFTSY | BGA13- |
| NO: 94 | WLHWVRQAPGQGLEWIGYINPQTGYTSYAQKF | 1F VH |
| | QGRVTMTRDTSTSTVYMELSSLRSEDTAVYYC | |
| | AREYGNYNYPLDYWGQGTLVTVSS | |

### Example 6. Generation of Affinity Maturation Libraries

A phagemid vector pCANTAB 5E (GE Healthcare) was used by standard molecular biology techniques to construct a phagemid designed to display BGA13-1F Fab fragments on the surface of M13 bacteriophage as a fusion with the N-terminus of a fragment of the gene-3 minor coat protein. There was an amber stop codon before the gene-3 sequence to allow expression of Fab fragments directly from phagemid clones. The phagemid was used as the template to construct phage-displayed libraries containing 108 unique members.

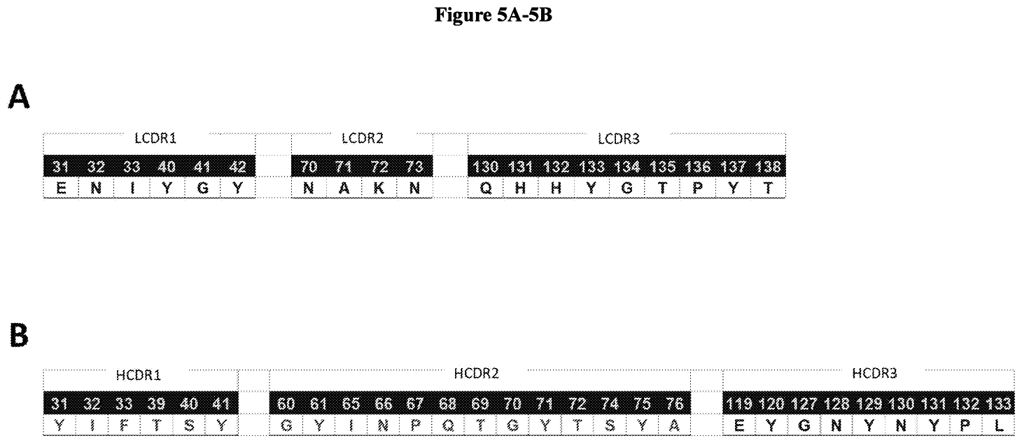

Two libraries (H-AM, L-AM) were constructed randomizing CDR positions in the heavy and light chains, respectively. All three CDRs were randomized in each library but each CDR had a maximum of one mutation in each clone except HCDR3, which could have two simultaneous mutations. Each position was randomized with an NNK codon (IUPAC code) encoding any amino acid or an amber stop codon. The combined heavy and light chain library designs had a potential diversity of 5.0×10⁶ unique full-length clones without stop or cysteine codons and an expected distribution of about 0.02%, 1.1%, 17% and 82% of clones with 0, 1, 2, and 3 mutations, respectively. A minor fraction of heavy chain clones was expected to have 4 mutations due to primer design in the HCDR3 region. As a first step, a DNA fragment was amplified using pCANTAB 5E as a template and primers which contains the randomized CDR3 positions (see FIGS. 5A and 5B). Then the PCR products were gel-purified and assembled with the primers which contains the randomized CDR2 positions. The procedure was repeated with the primers directed to random CDR1 positions. The resulting PCR products for heavy chain or light chain were then assembled with its corresponding CH fragment or CL fragment by overlapping PCR. The fragments were further assembled with the light chain or heavy chain with no mutations by overlapping PCR. The resulting fragments were then gel-purified and ligated with pCANTAB 5E after NcoI/NotI digestion. The purified ligations were transformed into TG1 bacteria by electroporation. Sequencing of 48 clones from each library confirmed the randomization of each position (data not shown), although not all amino acid mutations were observed in every position due to the limited sampling depth. About 52% and 55% of the light and heavy chain libraries had full-length randomized clones, enough to cover all the potential diversity of the design with the 108 independent clones generated even with moderate incorporation biases in oligonucleotide synthesis and library construction.

### Example 7. Generation of Affinity Matured Humanized BGA13 Variants

### Library Selection and Screening

Generation of affinity-matured humanized BGA13 Fabs was carried out by phage display using standard protocols (Silacci et al., (2005) Proteomics, 5, 2340-50; Zhao et al., (2014) PLOS One, 9, e111339). For the first and second rounds of selections, competition selections were performed on immobilized CHIM in immune tubes (Cat. No. 470319, ThermoFisher). In brief, immunotubes were coated with 1 ml of CHIM (5 μg/ml in PBS) overnight at 4° C. All affinity maturation libraries were incubated with the coated immunotubes for 1 hour in the presence of various concentrations of BGA13-1F IgG (round 1, 1 μg/ml; round 2, 5 μg/ml). For the third and fourth rounds of selections, cell panning was carried out using L929/huCEA cells (round 3) or LOVO cells (ATCC CCL-229) (round 4) with HEK293 cells as depletion cells. After four rounds of selections, individual clones were picked up and phage containing supernatants were prepared using standard protocols. ELISA-positive clones were sequenced, and mutation sites were analyzed.

### Analysis of Mutation Frequency in CDRs

The frequency of mutations in each CDR after four rounds of selection was relatively high, ranging from 17% in HCDR3 to 95% in LCDR2. Regarding the heavy chain, about half of clones identified in H-AM library were identical to the parental clone. The other clones contained one back-mutation at Q54N in HCDR2.

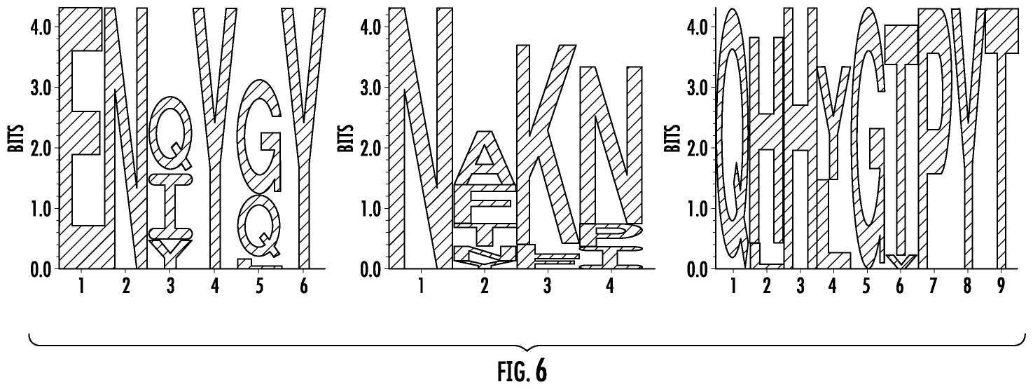

In analyzing the light chain, the mutations were much more diverse. Two sites had mutations occurring in almost all of clones in LCDR1, respectively. Light chain residues 29 and 31 were mutated from Ile to Gln and Gly to Gln in 47.09% and 35.29% of the clones, respectively. Position 29 not only had a high frequency of Gln mutation, but also had a subset of clones with a mutation to Tyrosine. Position 31 not only had a high frequency of Gln mutation, but also had about 12.5% chance to be mutated to Leu. Due to library design constraints, mutations in positions 29 and 31 were not found in combination with each other. However, mutations in each of these two sites were often combined with mutations in other CDRs. Regarding LCDR2, only A51 had mutations occurring in at least 64.71% clones, but with not any obvious pattern, which included large, hydrophobic and polar residues, such as Tyr, Phe, Thr and Asn. Regarding to LCDR3, two sites had mutations occurring in at least 50% clones. Light chain residues 90 and 92 were mutated form His to Leu and Tyr to Leu in 11.76% and 47.06% of the clones, respectively. FIG. 6 shows the sequence variance of CDR regions of light chain after four rounds of selections.

### Expression of Selected Humanized BGA13 Variants

Combination of mutations were made. Light chain variable regions from selected phage clones were subcloned into a human kappa light chain expression mammalian expression vector. The light chain expression vectors were co-transfected into 293G cells with a mammalian expression vector expressing BGA13-1F heavy chain at a 1:1 ratio. Versions of CEA antibodies were purified from culture supernatants by Protein A affinity chromatography (Cat. No. 17543802, GE Life Sciences). The purified antibodies were concentrated to 0.5-5 mg/mL in PBS and stored in aliquots in −80° C. freezer.

### Characterization of Affinity Matured Humanized BGA13 Variants

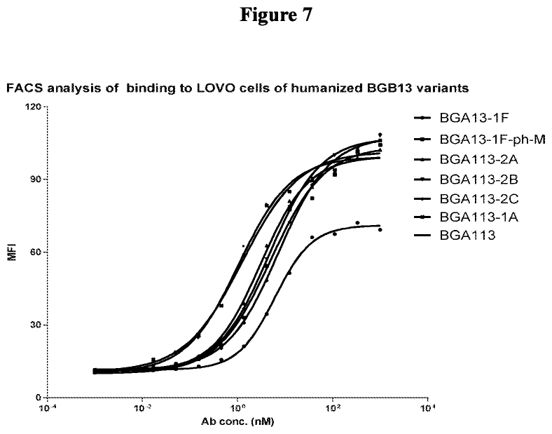

Affinity comparison of BGA13-1F and other affinity matured clones was made by SPR assay (Table 9) using BIAcore™ T-200 (GE Life Sciences) and flow cytometry (FIG. 7). For this experiment, anti-human IgG (Fc) antibody was immobilized on an activated CM5 biosensor chip (Cat. No. BR100839, GE Life Sciences). Anti-CEA antibodies were flowed over the chip surface and captured by anti-human Fab antibody. Then a serial dilution (1.37 nM to 333 nM) of CHIM was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). For flow cytometry, CEA-expressing cells (10⁵ cells/well) were incubated with various concentrations of purified affinity-matured antibodies, followed by binding with Alexa Fluro-647-labeled anti-hu IgG Fc antibody (Cat. No. 409320, BioLegend, USA). Cell fluorescence was quantified using a flow cytometer (Guava easyCyte™ 8HT, Merck-Millipore, USA). The equilibrium dissociation constant (K_{D}) was calculated as the ratio k_{off}/kₒₙ. The BGA131F-ph-L (SEQ ID NO: 95) and BGA131F-ph-M (SEQ ID NO: 96) were shown with improved affinities to huCEA surface protein (Table 10).

TABLE 9
Comparison of binding affinities by SPR

| Sample ID | Ka(1/Ms) | Kd(1/s) | KD(M) | Rmax(RU) |
|---|---|---|---|---|
| 1F-ph-E | 3.4E+3 | 3.5E−3 | 1.0E−6 | 284 |
| 1F-ph-F | 1.0E+2 | 1.2E−2 | 1.2E−4 | 4162 |
| 1F-ph-G | 6.4E+1 | 2.6E−3 | 4.1E−5 | 3487 |
| 1F-ph-H | 1.1E+2 | 2.8E−3 | 2.5E−5 | 1983 |
| 1F-ph-I | 2.0E+2 | 2.2E−3 | 1.1E−5 | 2248 |
| 1F-ph-L | 6.7E+3 | 3.0E−4 | 4.6E−8 | 184 |
| 1F-ph-M | 2.5E+3 | 2.5E−4 | 9.9E−8 | 224 |
| 1F-ph-N | 1.1E+3 | 1.0E−2 | 9.2E−6 | 1267 |
| BGA13-1F | 1.9E+3 | 1.5E−3 | 7.9E−7 | 367 |

TABLE 10
amino acid sequences

| | | |
|---|---|---|
| SEQ ID | DIQMTQSPSSLSASVGDRVTITCRASENQYGY | BGA13- |
| NO: 95 | LAWYQQKPGKVPKLLIYNFKNLVEGVPSRFSG | 1F-ph-L |
| | SGSGTDFTLTISSLQPEDVATYYCQHHLGTPY | VL |
| | TFGQGTKVEIK | |
| SEQ ID | DIQMTQSPSSLSASVGDRVTITCRASENQYGY | BGA13- |
| NO: 96 | LAWYQQKPGKVPKLLIYNTKNLVEGVPSRFSG | 1F-ph-M |
| | SGSGTDFTLTISSLQPEDVATYYCQHHLGTPY | VL |
| | TFGQGTKVEIK | |

### Example 8. Further Engineering of Affinity Matured Humanized BGA13 Variants

Further engineering was made by introducing mutations in CDRs based on BGA131F-ph-M template, which included W33Y, Q54N and S59N in the VH and T51Y in the VL. This resulted in BGA1132A (W33Y (VH)), BGA1132B (Q54N (VH)), BGA 1132C (S59N (VH)), BGA 1131A (T51Y (VL)) which all had improved binding activities to BGA-1131F, with the most improved antibody finally resulting in the BGA113 antibody with the (W33Y (VH), T51Y (VL)) changes (Table 11), with the sequences shown in Table 12.

TABLE 11
Comparison of binding affinities to CHIM by SPR

| Sample ID | Ka(1/Ms) | Kd(1/s) | KD(M) | Rmax(RU) |
|---|---|---|---|---|
| BGA1311F-ph-M | 1.97E+04 | 2.48E−04 | 1.26E−08 | 97.2 |
| 1132A | 1.99E+04 | 2.82E−04 | 1.42E−08 | 129.1 |
| 1132B | 1.98E+04 | 3.80E−04 | 1.92E−08 | 71.1 |
| 1132C | 1.84E+04 | 3.94E−04 | 2.15E−08 | 59.4 |
| 1131A | 3.21E+04 | 4.63E−04 | 1.44E−08 | 92.9 |
| 113 | 3.15E+04 | 4.75E−04 | 1.51E−08 | 112.4 |

TABLE 12
Amino acid and nucleic acid sequences of BGA113

| | | | |
|---|---|---|---|
| BGA113 | SEQ ID | VH | QVQLVQSGAEVKKPGASVKVSCKASGYIF |
| | NO: 97 | | TSYYLHWVRQAPGQGLEWIGYINPQTGY |
| | | | TSYAQKFQGRVTMTRDTSTSTVYMELSSL |
| | | | RSEDTAVYYCAREYGNYNYPLDYWGQGT |
| | | | LVTVSS |
| | SEQ ID | VL | DIQMTQSPSSLSASVGDRVTITCRASENQ |
| | NO: 15 | | YGYLAWYQQKPGKVPKLLIYNYKNLVEGV |
| | | | PSRFSGSGSGTDFTLTISSLQPEDVATYY |
| | | | CQHHLGTPYTFGQGTKVEIK |
| | SEQ ID | VH DNA | CAGGTGCAGCTGGTGCAGAGCGGCGCG |
| | NO: 98 | | GAAGTGAAAAAACCGGGCGCGAGCGTG |
| | | | AAAGTGAGCTGCAAAGCGAGCGGCTATA |
| | | | TTTTTACCAGCTATTACCTGCATTGGGTG |
| | | | CGCCAGGCGCCGGGCCAGGGCCTGGAAT |
| | | | GGATTGGCTATATTAACCCGCAGACCGGC |
| | | | TATACCAGCTATGCCCAGAAATTTCAGGG |
| | | | CCGCGTGACCATGACCCGCGATACCAGC |
| | | | ACCAGCACCGTGTATATGGAACTGAGCA |
| | | | GCCTGCGCAGCGAAGATACCGCGGTGTA |
| | | | TTATTGCGCGCGCGAATATGGCAACTATA |
| | | | ACTATCCGCTGGATTATTGGGGCCAGGGC |
| | | | ACCCTGGTGACCGTGAGCAGC |
| | SEQ ID | VL DNA | GATATTCAGATGACCCAGAGCCCGAGCA |
| | NO: 17 | | GCCTGAGCGCGAGCGTGGGCGATCGCGT |
| | | | GACCATTACCTGCCGCGCGAGCGAAAAC |
| | | | CAGTATGGCTATCTGGCGTGGTATCAGCA |
| | | | GAAACCGGGCAAAGTGCCGAAACTGCT |
| | | | GATTTATAACTATAAAAACCTGGTGGAAG |
| | | | GCGTGCCGAGCCGCTTTAGCGGCAGCGG |
| | | | CAGCGGCACCGATTTTACCCTGACCATTA |
| | | | GCAGCCTGCAGCCGGAAGATGTGGCGAC |
| | | | CTATTATTGCCAGCATCATCTGGGCACCC |
| | | | CGTATACCTTTGGCCAGGGCACCAAAGT |
| | | | GGAAATTAAA |

### Example 9. Optimization of BGA113

To further improve the biochemical/biophysical properties, optimization of BGA113 was made by introducing substitutions in CDR and framework regions (Table 13). The large, hydrophobic residues were chosen and changed to polar residues, except for K13 and Q53, which are selected based on observed differences among human VH germlines. The considerations include amino acid compositions, heat stability (Tm), surface hydrophobicity and isoelectronic points (pIs) while maintaining functional activities. The variants were expressed in Fab format by cloning into the vector pCANTAB-5E as described in Example 6. The Fab-containing supernatants were then screened by ELISA and SPR analysis for CEA binding. The variants without significant affinity reduction were selected and the residues which can tolerate substitutions were identified. It was demonstrated L92E in the light chain, K13E, Q54E, Y57D/E and Y57K in the heavy chain have minimal influence on the affinity. Thus, the BGA113 variants in IgG format with single identified mutation or combinations were expressed and purified as described in Example 8. SPR study and FACS analysis were performed and summarized in Table 14. It was confirmed that no changes in specificity and epitope occurred due to the introduced amino acid substitutions (data not shown). Taken together, the results demonstrated these single or combined mutations (K13E, Q54E, Y57D and Y57K in the heavy chain, L92E in the light chain) have minimal effects on the affinity, except for L92E, which slightly reduces the binding affinity to CEA. In summary, the Y57K change optimized the BGA113 antibody for expression, CEA binding and affinity, which resulted in BGA113K (Table 1).

TABLE 13
Summary of residues for substitution

| Residue | AA substitution |
|---|---|
| H: K13 | E |
| H: Y32 | H, N, Q, D, E, K |
| H: Y33 | H, N, Q, D, E, K |
| H: Q53 | A, D, G, N, S, T, Y, R, H, |
| H: Y57 | H, N, Q, D, E, K |
| H: Y100 | H, N, Q, D, E, K |
| H: Y105 | H, N, Q, D, E, K |
| L: V15 | T, P, L |
| L: Y30 | H, N, Q, D, E, K |
| L: Y32 | H, N, Q, D, E, K |
| L: Y49 | H, N, Q, D, E, K |
| L: P80 | S, T, A |
| L: L92 | H, N, Q, D, E, K |

TABLE 14
Summary of affinity measurement of BGA113 variants by SPR

| | | | | Affinity | |
|---|---|---|---|---|---|
| | Kinetics (huCEA) | | | (MKN45 cells) | |
| BGA113 | ka | kd | KD | | EC50 |
| variants | (1/Ms) | (1/s) | (M) | Top | (ug/ml) |
| L92E, Y57K | 3.34E+04 | 1.77E−04 | 5.29E−09 | 969.9 | 1.09 |
| L92E | 4.17E+04 | 1.88E−04 | 4.50E−09 | 945.4 | 1.19 |
| Q54E, Y57K | 1.10E+05 | 2.60E−04 | 2.36E−09 | 901.9 | 0.58 |
| Y57K | 1.02E+05 | 1.81E−04 | 1.77E−09 | 934.6 | 0.52 |
| K13E, Y57K | 1.28E+05 | 1.72E−04 | 1.34E−09 | 959.9 | 0.48 |
| Q54E, Y57E | 1.17E+05 | 2.50E−04 | 2.14E−09 | 926.9 | 0.65 |
| Y57D | 1.07E+05 | 1.95E−04 | 1.82E−09 | 949.3 | 0.56 |
| BGA113 | 1.13E+05 | 1.69E−04 | 1.49E−09 | 919.3 | 0.49 |

### Example 10. Binding Profiles of Anti-CEA Antibody BGA113K

BGA113K and a previously disclosed CEA antibody, designated as antibody 2F1 in U.S. 2012/0251529, were generated in human IgG1 format and characterized for their binding kinetics by SPR assays using BIAcore™ T-200 (GE Life Sciences).

To obtain this data, anti-human IgG (Fc) antibody was immobilized on an activated CM5 biosensor chip (Cat. No. BR100839, GE Life Sciences). The BGA113K antibody was flowed over the chip surface and captured by anti-human Fab antibody. Then a serial dilution (1.37 nM to 2150 nM) of soluble huCEA or cynoCEA(Cat.: CE5-C52H5, Acrobiosystem) were flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. BGA113K and and the 2F1 control antibody displayed different binding affintiy. BGA113K has very high affinity for human CEA and also a comparable affinity for cynoCEA, as shown in Table 15 below.

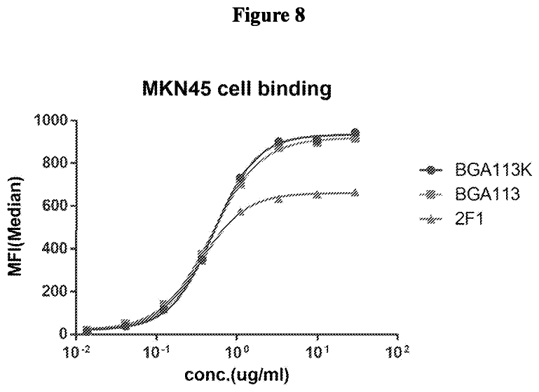

For flow cytometry, CEA-expressing MKN45 cells (10⁵ cells/well) were incubated with various concentrations of purified affinity-matured antibodies, followed by binding with Alexa Fluro-647-labeled anti-hu IgG Fc antibody (Cat. No. 409320, BioLegend, USA). Cell fluorescence was quantified using a flow cytometer (Guava easyCyte™ 8HT, Merck-Millipore, USA). As shown in FIG. 8, BGA-113K demonstrated specific binding to native CEA on living cells in a dose-responsive manner with EC50 of 2.92 μg/ml.

TABLE 15
Comparison of binding affinities
of anti-CEA antibodies by SPR

| | BGA113K | | | 2F1 Antibody | | |
|---|---|---|---|---|---|---|
| | Ka | Kd | KD | Ka | Kd | KD |
| | (1/Ms) | (1/s) | (M) | (1/Ms) | (1/s) | (M) |
| huCEA | 1.02E+ | 1.81E− | 1.77E− | 4.50E+ | 3.00E− | 6.50E− |
| | 05 | 04 | 09 | 4 | 3 | 8 |
| cynoCEA | 2.67E+ | 5.34E− | 1.99E− | ND | ND | ND |
| | 04 | 04 | 08 | | | |

### Example 11. Evaluation of Off-Target Specificity

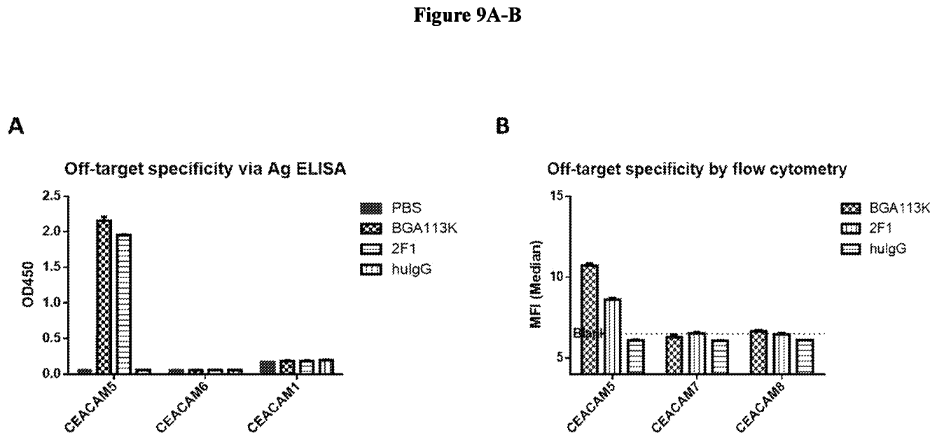

The off-target specificity of BGA113K was evaluated via ELISA and flow cytometry. For flow cytometry CEACAM3 (SEQ ID NO:65), CEACAM7 (SEQ ID NO:66) or CEACAM8 (SEQ ID NO:67) were transiently transfected into HEK293 cells (10⁵ cells/well) and then were incubated with 2 μg/ml purified BGA113K, followed by binding with Alexa Fluor-647-labeled anti-huIgG Fc antibody (Cat. No. 409320, BioLegend, USA). Cell fluorescence was quantified using a flow cytometer (Guava easyCyte™ 8HT, Merck-Millipore, USA). For antigen ELISA, CEACAM1(SEQ ID NO:64) (Cat. No. 10822-H08H, Sino Biological, China), CHIM (SEQ ID NO: 63), CEA (SEQ ID NO:55) or CEACAM6 (SEQ ID NO:57) were coated in 96-well plates at a concentration of 10 μg/ml overnight at 4° C. The HRP-linked anti-human Fc (Fc specific) IgG antibody (Cat. No. A0170, Sigma, USA) and substrate (Cat. No. 00-4201-56, eBioscience, USA) were used for development, and absorbance signal at the wavelength of 450 nm was measured using a plate reader (SpectraMax Paradigm, Molecular Devices, USA). As shown in FIGS. 9A-9B, no cross-reactivity to other CEACAM family members were observed, thus BGA113K demonstrated specificity only for CEA (CEACAM5 in FIGS. 9A-B).

### Example 12. Effects of Soluble huCEA on Binding of BGA113K to CEA Expressing Cells

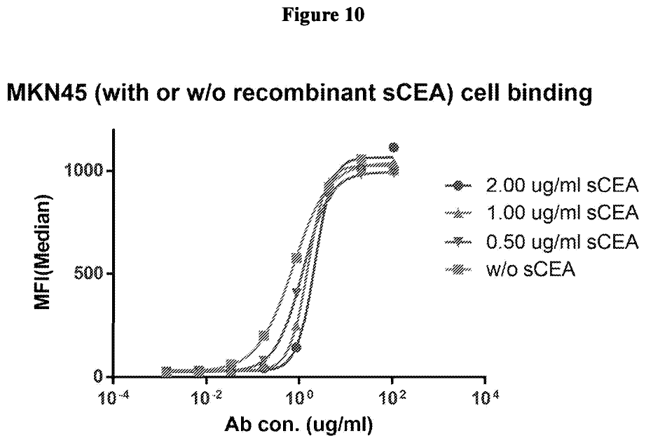

To determine if soluble CEA (sCEA) had any effect on the specific binding of BGA113K, various concentrations (0, 0.5, 1, 2 g/ml) of recombinant soluble CEA was premixed with (0.01 ˜100 μg/ml) BGA113K and incubated for 5 min. The mixtures were then incubated with 2×10⁵ CEA expressing cells, such as MKN45 cells for 30 min at 4° C. The cells were stained with secondary antibody anti-huFc-APC (Cat. No. 409320, BioLegend, USA) and analyzed by flow cytometry. In the presence of 2 μg/ml of recombinant sCEA, the binding of BGA113K to CEA expressing cells was not affected. This result is shown for MKN45 cells (FIG. 10) and indicates BGA113K's specificity for the membrane bound form of CEA.

### Example 13. BGA113 Induces Potent ADCC Effects on CEA⁺ Tumor Cells

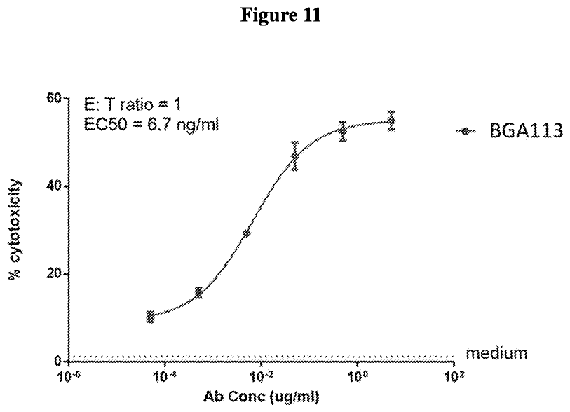

To determine whether BGA113 in the wild-type IgG1 format can induce antibody dependent cytotoxicity (ADCC), CD16(V158)-expressing NK92MI cells (NK92MI/CD16V) were used as effector cells and were co-cultured with mouse colon cancer cells (CT26-ATCC CRL-2638) expressing CEA. The co-culture was performed at an E:T ratio of 1:1 for 5 hours in the presence of BGA113 at indicated concentrations (0.00005-5 μg/ml), and cytotoxicity was determined by Lactate dehydrogenase (LDH) release. The amounts of LDH in the supernatant were measured using the CytoTox™ 96 Non-Radioactive Cytotoxicity Assay kit (Promega, Madison, WI), and the amount of specific lysis was calculated according to the manufacturer's instruction. As shown in FIG. 11, BGA113 could induce ADCC in vitro with an EC₅₀ around 6.7 ng/ml.

### Example 14. In vivo anti-tumor efficacy of BGA113

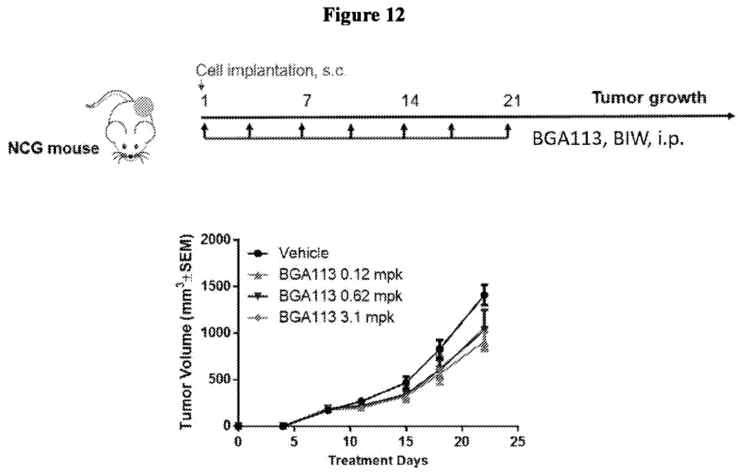

To determine the in vivo efficacy of BGA113 against CEA tumor cells, NK92MI/CD16V cells (5×10⁶) were mixed with CT26/CEA cells (10⁶) and injected subcutaneously into NCG mice. BGA113 (0.12, 0.62 or 3.1 mg/kg) or vehicle control was given twice a week starting on the day of tumor injection (7 mice per group). As compared to vehicle, BGA113 at 3.1 mg/kg dosage showed a low amount of tumor inhibition, although the difference from vehicle control was not statistically significant (P>0.05) (FIG. 12).

### REFERENCES

Abdul-Wahid, A., M. Cydzik, N. W. Fischer, A. Prodeus, J. E. Shively, A. Martel, S. Alminawi, Z. Ghorab, N. L. Berinstein & J. Gariépy (2018) Serum-derived carcinoembryonic antigen (CEA) activates fibroblasts to induce a local re-modeling of the extracellular matrix that favors the engraftment of CEA-expressing tumor cells. International Journal of Cancer, 143, 1963-1977.
Abdul-Wahid, A., E. H. B. Huang, M. Cydzik, E. Bolewska-Pedyczak & J. Gariépy (2014) The carcinoembryonic antigen IgV-like N domain plays a critical role in the implantation of metastatic tumor cells. Molecular Oncology, 8, 337-350.
Augugliaro, R., S. Parolini, R. Castriconi, E. Marcenaro, C. Cantoni, M. Nanni, L. Moretta, A. Moretta & C. Bottino (2003) Selective cross-talk among natural cytotoxicity receptors in human natural killer cells. Eur J Immunol, 33, 1235-41.
Bacac, M., C. Klein & P. Umana (2016) CEA TCB: A novel head-to-tail 2:1 T cell bispecific antibody for treatment of CEA-positive solid tumors. Oncoimmunology, 5, e1203498.
Barrow, A. D., C. J. Martin & M. Colonna (2019) The Natural Cytotoxicity Receptors in Health and Disease. Front Immunol, 10, 909.
Beauchemin, N. 2017. CEA Gene Family. In Encyclopedia of Cancer, ed. M. Schwab, 870-874. Berlin, Heidelberg: Springer Berlin Heidelberg.
Beck, A., T. Wurch, C. Bailly & N. Corvaia (2010) Strategies and challenges for the next generation of therapeutic antibodies. Nat Rev Immunol, 10, 345-52.
Biron, C. A., K. B. Nguyen, G. C. Pien, L. P. Cousens & T. P. Salazar-Mather (1999) Natural killer cells in antiviral defense: function and regulation by innate cytokines. Annu Rev Immunol, 17, 189-220.
Brocks, B., P. Garin-Chesa, E. Behrle, J. E. Park, W. J. Rettig, K. Pfizenmaier & D. Moosmayer (2001) Species-crossreactive scFv against the tumor stroma marker “fibroblast activation protein” selected by phage display from an immunized FAP−/− knock-out mouse. Molecular medicine (Cambridge, Mass.), 7, 461-469.
Chau, I., M. J. Allen, D. Cunningham, A. R. Norman, G. Brown, H. E. Ford, N. Tebbutt, D. Tait, M. Hill, P. J. Ross & J. Oates (2004) The value of routine serum carcino-embryonic antigen measurement and computed tomography in the surveillance of patients after adjuvant chemotherapy for colorectal cancer. J Clin Oncol, 22, 1420-9.
de St Groth, S. F. & D. Scheidegger (1980) Production of monoclonal antibodies: strategy and tactics. J Immunol Methods, 35, 1-21.
Dotan, E., A. Starodub, J. Berlin, C. H. Lieu, M. J. Guarino, J. Marshall, J. R. Hecht, S. J. Cohen, W. A. Messersmith, P. P. Maliakal, W. A. Wegener, R. M. Sharkey & D. M. Goldenberg (2015) A new anti-CEA-SN-38 antibody-drug conjugate (ADC), IMMU-130, is active in controlling metastatic colorectal cancer (mCRC) in patients (pts) refractory or relapsing after irinotecan-containing chemotherapies: Initial results of a phase I/II study. Journal of Clinical Oncology, 33, 2505-2505.
Durbin, H., S. Young, L. M. Stewart, F. Wrba, A. J. Rowan, D. Snary & W. F. Bodmer (1994) An epitope on carcinoembryonic antigen defined by the clinically relevant antibody PR1A3. Proc Natl Acad Sci USA, 91, 4313-7.
Engler, F. A., J. R. Polli, T. Li, B. An, M. Otteneder, J. Qu & J. P. Balthasar (2018) “Catch-and-Release” Anti-Carcinoembryonic Antigen Monoclonal Antibody Leads to Greater Plasma and Tumor Exposure in a Mouse Model of Colorectal Cancer. J Pharmacol Exp Ther, 366, 205-219.
Fischer, E., K. Chaitanya, T. Wuest, A. Wadle, A. M. Scott, M. van den Broek, R. Schibli, S. Bauer & C. Renner (2012) Radioimmunotherapy of fibroblast activation protein positive tumors by rapidly internalizing antibodies. Clin Cancer Res, 18, 6208-18.
Flamini, E., L. Mercatali, O. Nanni, D. Calistri, R. Nunziatini, W. Zoli, P. Rosetti, N. Gardini, A. Lattuneddu, G. M. Verdecchia & D. Amadori (2006) Free DNA and carcinoembryonic antigen serum levels: an important combination for diagnosis of colorectal cancer. Clin Cancer Res, 12, 6985-8.
Gangopadhyay, A., D. A. Lazure & P. Thomas (1997) Carcinoembryonic antigen induces signal transduction in Kupffer cells. Cancer Letters, 118, 1-6.
Gauthier, L., A. Morel, N. Anceriz, B. Rossi, A. Blanchard-Alvarez, G. Grondin, S. Trichard, C. Cesari, M. Sapet, F. Bosco, H. Rispaud-Blanc, F. Guillot, S. Cornen, A. Roussel, B. Amigues, G. Habif, F. Caraguel, S. Arrufat, R. Remark, F. Romagne, Y. Morel, E. Narni-Mancinelli & E. Vivier (2019) Multifunctional Natural Killer Cell Engagers Targeting NKp46 Trigger Protective Tumor Immunity. Cell, 177, 1701-1713 e16.
Gold, P. & S. O. Freedman (1965) Demonstration of tumor-specific antigens in human colonic carcinomata by immunological tolerance and absorption techniques. J Exp Med, 121, 439-62.
Goldenberg, D. M. (1992) Cancer imaging with CEA antibodies: historical and current perspectives. Int J Biol Markers, 7, 183-8.
Hammarström, S. (1999) The carcinoembryonic antigen (CEA) family: structures, suggested functions and expression in normal and malignant tissues. Seminars in Cancer Biology, 9, 67-81.
Hezareh, M., A. J. Hessell, R. C. Jensen, J. G. van de Winkel & P. W. Parren (2001) Effector function activities of a panel of mutants of a broadly neutralizing antibody against human immunodeficiency virus type 1. J Virol, 75, 12161-8.
Hohenberger, P., P. M. Schlag, T. Gerneth & C. Herfarth (1994) Pre- and postoperative carcinoembryonic antigen determinations in hepatic resection for colorectal metastases. Predictive value and implications for adjuvant treatment based on multivariate analysis. Ann Surg, 219, 135-43.
Hudspeth, K., B. Silva-Santos & D. Mavilio (2013) Natural cytotoxicity receptors: broader expression patterns and functions in innate and adaptive immune cells. Front Immunol, 4, 69.
Hurwitz, E., I. Stancovski, M. Sela & Y. Yarden (1995) Suppression and promotion of tumor growth by monoclonal antibodies to ErbB-2 differentially correlate with cellular uptake. Proc Natl Acad Sci USA, 92, 3353-7.
Igawa, T., H. Tsunoda, Y. Kikuchi, M. Yoshida, M. Tanaka, A. Koga, Y. Sekimori, T. Orita, Y. Aso, K. Hattori & M. Tsuchiya (2010) V_{H}/VL interface engineering to promote selective expression and inhibit conformational isomerization of thrombopoietin receptor agonist single-chain diabody. Protein Eng Des Sel, 23, 667-77.
Koch, J., A. Steinle, C. Watzl & O. Mandelboim (2013) Activating natural cytotoxicity receptors of natural killer cells in cancer and infection. Trends Immunol, 34, 182-91.
Kuespert, K., S. Pils & C. R. Hauck (2006) CEACAMs: their role in physiology and pathophysiology. Current Opinion in Cell Biology, 18, 565-571.
Kruse, P. H., J. Matta, S. Ugolini & E. Vivier (2014) Natural cytotoxicity receptors and their ligands. Immunol Cell Biol, 92, 221-9.
Lanier, L. L. (2015) NKG2D Receptor and Its Ligands in Host Defense. Cancer Immunol Res, 3, 575-82.
Lee, J. H. & S.-W. Lee (2017) The Roles of Carcinoembryonic Antigen in Liver Metastasis and Therapeutic Approaches. Gastroenterology Research and Practice, 2017, 11.
Lee, K.-A., E.-A. Bae, Y. C. Song, E.-K. Kim, Y.-S. Lee, T.-G. Kim & C.-Y. Kang (2015) A multimeric carcinoembryonic antigen signal inhibits the activation of human T cells by a SHP-independent mechanism: A potential mechanism for tumor-mediated suppression of T-cell immunity. International Journal of Cancer, 136, 2579-2587.
Lefranc, M. P., V. Giudicelli, C. Ginestoux, J. Bodmer, W. Muller, R. Bontrop, M. Lemaitre, A. Malik, V. Barbie & D. Chaume (1999) IMGT, the international ImMunoGeneTics database. Nucleic Acids Res, 27, 209-12.
Liersch, T., J. Meller, M. Bittrich, B. Kulle, H. Becker & D. M. Goldenberg (2007) Update of carcinoembryonic antigen radioimmunotherapy with (131)I-labetuzumab after salvage resection of colorectal liver metastases: comparison of outcome to a contemporaneous control group. Ann Surg Oncol, 14, 2577-90.
Liu, L., W. Zeng, M. A. Wortinger, S. B. Yan, P. Cornwell, V. L. Peek, J. R. Stephens, J. W. Tetreault, J. Xia, J. R. Manro, K. M. Credille, D. W. Ballard, P. Brown-Augsburger, V. Wacheck, C. K. Chow, L. Huang, Y. Wang, I. Denning, J. Davies, Y. Tang, P. Vaillancourt & J. Lu (2014) LY2875358, a neutralizing and internalizing anti-MET bivalent antibody, inhibits HGF-dependent and HGF-independent MET activation and tumor growth. Clin Cancer Res, 20, 6059-70.
Matsuoka, Y., Y. Matsuo, K. Sugano, H. Ohkura, M. Kuroki & M. Kuroki (1990) Characterization of Carcinoembryonic Antigen-related Antigens in Normal Adult Feces. Japanese Journal of Cancer Research, 81, 514-519.
Mechetner, E. (2007) Development and characterization of mouse hybridomas. Methods Mol Biol, 378, 1-13.
Moertel, C. G., T. R. Fleming, J. S. Macdonald, D. G. Haller, J. A. Laurie & C. Tangen (1993) An evaluation of the carcinoembryonic antigen (CEA) test for monitoring patients with resected colon cancer. Jama, 270, 943-7.
Nap, M., M. L. Hammarstrom, O. Bormer, S. Hammarstrom, C. Wagener, S. Handt, M. Schreyer, J. P. Mach, F. Buchegger, S. von Kleist & et al., (1992) Specificity and affinity of monoclonal antibodies against carcinoembryonic antigen. Cancer Res, 52, 2329-39.
Nap, M., K. Mollgard, P. Burtin & G. J. Fleuren (1988) Immunohistochemistry of carcino-embryonic antigen in the embryo, fetus and adult. Tumour Biol, 9, 145-53.
Oberst, M. D., S. Fuhrmann, K. Mulgrew, M. Amann, L. Cheng, P. Lutterbuese, L. Richman, S. Coats, P. A. Baeuerle & S. A. Hammond (2014) CEA/CD3 bispecific antibody MEDI-565/AMG 211 activation of T cells and subsequent killing of human tumors is independent of mutations commonly found in colorectal adenocarcinomas. MAbs, 6, 1571-84.
Pegram, H. J., D. M. Andrews, M. J. Smyth, P. K. Darcy & M. H. Kershaw (2011) Activating and inhibitory receptors of natural killer cells. Immunol Cell Biol, 89, 216-24.
Peng, L., M. D. Oberst, J. Huang, P. Brohawn, C. Morehouse, K. Lekstrom, P. A. Baeuerle, H. Wu, Y. Yao, S. R. Coats, W. Dall'Acqua, M. Damschroder & S. A. Hammond (2012) The CEA/CD3-bispecific antibody MEDI-565 (MT111) binds a nonlinear epitope in the full-length but not a short splice variant of CEA. PloS one, 7, e36412-e36412.
Poul, M. A., B. Becerril, U. B. Nielsen, P. Morisson & J. D. Marks (2000) Selection of tumor-specific internalizing human antibodies from phage libraries. J Mol Biol, 301, 1149-61.
Primrose, J. N., R. Perera, A. Gray, P. Rose, A. Fuller, A. Corkhill, S. George & D. Mant (2014) Effect of 3 to 5 years of scheduled CEA and CT follow-up to detect recurrence of colorectal cancer: the FACS randomized clinical trial. Jama, 311, 263-70.
Richman, P. I. & W. F. Bodmer (1987) Monoclonal antibodies to human colorectal epithelium: markers for differentiation and tumour characterization. Int J Cancer, 39, 317-28.
Ridgway, J. B., L. G. Presta & P. Carter (1996) ‘Knobs-into-holes’ engineering of antibody CH3 domains for heavy chain heterodimerization. Protein Eng, 9, 617-21.
Saito, G., S. Sadahiro, K. Okada, A. Tanaka, T. Suzuki & A. Kamijo (2016) Relation between Carcinoembryonic Antigen Levels in Colon Cancer Tissue and Serum Carcinoembryonic Antigen Levels at Initial Surgery and Recurrence. Oncology, 91, 85-9.
Sakurai, Y., S. Hirohashi, T. Ohishi, Y. Shimosato, S. Kodaira & O. Abe (1989) Conformational epitopes specific to carcinoembryonic antigen defined by monoclonal antibodies raised against colon cancer xenografts. J Surg Oncol, 42, 39-46.
Sheahan, K., M. J. O'Brien, B. Burke, P. A. Dervan, J. C. O'Keane, L. S. Gottlieb & N. Zamcheck (1990) Differential reactivities of carcinoembryonic antigen (CEA) and CEA-related monoclonal and polyclonal antibodies in common epithelial malignancies. Am J Clin Pathol, 94, 157-64.
Shinmi, D., R. Nakano, K. Mitamura, M. Suzuki-Imaizumi, J. Iwano, Y. Isoda, J. Enokizono, Y. Shiraishi, E. Arakawa, K. Tomizuka & K. Masuda (2017) Novel anticarcinoembryonic antigen antibody-drug conjugate has antitumor activity in the existence of soluble antigen. Cancer Med, 6, 798-808.
Silacci, M., S. Brack, G. Schirru, J. Marlind, A. Ettorre, A. Merlo, F. Viti & D. Neri (2005) Design, construction, and characterization of a large synthetic human antibody phage display library. Proteomics, 5, 2340-50.
Stern, N., G. Markel, T. I. Arnon, R. Gruda, H. Wong, S. D. Gray-Owen & O. Mandelboim
(2005) Carcinoembryonic Antigen (CEA) Inhibits NK Killing via Interaction with CEA-Related Cell Adhesion Molecule 1. The Journal of Immunology, 174, 6692-6701.
Stewart, L. M., S. Young, G. Watson, S. J. Mather, P. A. Bates, H. A. Band, R. W. Wilkinson, E. L. Ross & D. Snary (1999) Humanisation and characterisation of PRIA3, a monoclonal antibody specific for cell-bound carcinoembryonic antigen. Cancer Immunol Immunother, 47, 299-306.
Takahashi, T., S. Minami, Y. Tsuchiya, K. Tajima, N. Sakai, K. Suga, S. I. Hisanaga, N. Ohbayashi, M. Fukuda & H. Kawahara (2019) Cytoplasmic control of Rab family small GTPases through BAG6. EMBO Rep, 20.
Thompson, J. A., F. Grunert & W. Zimmermann (1991) Carcinoembryonic antigen gene family: Molecular biology and clinical perspectives. Journal of Clinical Laboratory Analysis, 5, 344-366.
Wong, J. Y., D. Z. Chu, L. E. Williams, D. M. Yamauchi, D. N. Ikle, C. S. Kwok, A. Liu, S. Wilczynski, D. Colcher, P. J. Yazaki, J. E. Shively, A. M. Wu & A. A. Raubitschek (2004) Pilot trial evaluating an 123I-labeled 80-kilodalton engineered anticarcinoembryonic antigen antibody fragment (cT84.66 minibody) in patients with colorectal cancer. Clin Cancer Res, 10, 5014-21.
Wu, J., Y. Song, A. B. Bakker, S. Bauer, T. Spies, L. L. Lanier & J. H. Phillips (1999) An activating immunoreceptor complex formed by NKG2D and DAP10. Science, 285, 730-2.
Wu, T. T. & E. A. Kabat (1970) An analysis of the sequences of the variable regions of Bence Jones proteins and myeloma light chains and their implications for antibody complementarity. J Exp Med, 132, 211-50.
Zhang, T., B. A. Lemoi & C. L. Sentman (2005) Chimeric NK-receptor-bearing T cells mediate antitumor immunotherapy. Blood, 106, 1544-1551.
Zhang, T., X. Song, L. Xu, J. Ma, Y. Zhang, W. Gong, Y. Zhang, X. Zhou, Z. Wang, Y. Wang, Y. Shi, H. Bai, N. Liu, X. Yang, X. Cui, Y. Cao, Q. Liu, J. Song, Y. Li, Z. Tang, M. Guo, L. Wang & K. Li (2018) The binding of an anti-PD-1 antibody to FcgammaRIota has a profound impact on its biological functions. Cancer Immunol Immunother, 67, 1079-1090.
Zhao, L., L. Qu, J. Zhou, Z. Sun, H. Zou, Y. Y. Chen, J. D. Marks & Y. Zhou (2014) High throughput identification of monoclonal antibodies to membrane bound and secreted proteins using yeast and phage display. PLOS One, 9, e111339.

1. An anti-CEA antibody or antigen-binding fragment thereof, comprising an antibody or antigen-binding fragment thereof that specifically binds to human CEA at amino acids 596 to 674 of SEQ ID NO:52.

2. The anti-CEA antibody or antigen-binding fragment thereof of claim 1, wherein the antibody or antigen-binding fragment thereof does not bind to other CEACAM family members.

3. The anti-CEA antibody or antigen-binding fragment thereof of claim 1, wherein the antibody or antigen-binding fragment thereof comprises:
(i) a heavy chain variable region that comprises (a) a HCDR1 of SEQ ID NO: 7, (b) a HCDR2 of SEQ ID NO:8, and (c) a HCDR3 of SEQ ID NO:9; and a light chain variable region that comprises: (d) a LCDR1 of SEQ ID NO: 10, (e) a LCDR2 of SEQ ID NO: 11, and (f) a LCDR3 of SEQ ID NO:6;
(ii) a heavy chain variable region that comprises (a) a HCDR1 of SEQ ID NO:24, (b) a HCDR2 of SEQ ID NO:25, and (c) a HCDR3 of SEQ ID NO:26; and a light chain variable region that comprises: (d) a LCDR1 of SEQ ID NO:27, (e) a LCDR2 of SEQ ID NO:28, and (f) a LCDR3 of SEQ ID NO: 23; or
(iii) a heavy chain variable region that comprises (a) a HCDR1 of SEQ ID NO:41, (b) a HCDR2 of SEQ ID NO:42, and (c) a HCDR3 of SEQ ID NO:43; and a light chain variable region that comprises: (d) a LCDR1 of SEQ ID NO:44, (e) a LCDR2 of SEQ ID NO:45, and (f) a LCDR3 of SEQ ID NO:40.

4. The anti-CEA antibody or antigen-binding fragment thereof of claim 3, wherein the antibody or antigen-binding fragment thereof comprises:
(i) a heavy chain variable region comprising an amino acid sequence at least 90% identical to SEQ ID NO: 14, and a light chain variable region comprising an amino acid sequence at least 90% identical to SEQ ID NO: 15;
(ii) a heavy chain variable region comprising an amino acid sequence at least 90% identical to SEQ ID NO: 31, and a light chain variable region comprising an amino acid sequence at least 90% identical to SEQ ID NO: 32; or
(iii) a heavy chain variable region comprising an amino acid sequence at least 90% identical to SEQ ID NO: 48, and a light chain variable region comprising an amino acid sequence at least 90% identical to SEQ ID NO: 49.

5. The anti-CEA antibody or antigen-binding fragment thereof of claim 4, wherein one, two, three, four, five, six, seven, eight, nine, or ten amino acids within SEQ ID NO:14, 15, 31, 32, 48, or 49 have been inserted, deleted or substituted.

6. The anti-CEA antibody or antigen-binding fragment thereof of claim 1, wherein the antibody or antigen-binding fragment thereof comprises:
(i) a heavy chain variable region that comprises SEQ ID NO:14, and a light chain variable region (VL) that comprises SEQ ID NO: 15;
(ii) a heavy chain variable region that comprises SEQ ID NO: 31, and a light chain variable region (VL) that comprises SEQ ID NO: 32; or
(iii) a heavy chain variable region that comprises SEQ ID NO: 48, and a light chain variable region (VL) that comprises SEQ ID NO: 49.

7. The anti-CEA antibody or antigen-binding fragment thereof of claim 1, wherein the anti-CEA antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

8. The anti-CEA antibody or antigen-binding fragment thereof of claim 1, wherein the antibody or antigen-binding fragment thereof has one or more of antibody dependent cellular cytotoxicity (ADCC) or complement dependent cytotoxicity (CDC).

9. The anti-CEA antibody or antigen-binding fragment thereof of claim 1, wherein the antibody or antigen-binding fragment thereof has reduced glycosylation or no glycosylation or is hypofucosylated.

10. The anti-CEA antibody or antigen-binding fragment thereof of claim 1, wherein the antibody or antigen-binding fragment thereof comprises increased bisecting GlcNac structures.

11. The anti-CEA antibody or antigen-binding fragment thereof of claim 1, wherein the Fc domain is an IgG1.

12. The anti-CEA antibody or antigen-binding fragment thereof of claim 1, wherein the antibody or antigen-binding fragment thereof is conjugated to a toxin.

13. A pharmaceutical composition comprising the anti-CEA antibody or antigen-binding fragment thereof of claim 1, and a pharmaceutically acceptable carrier.

14. A method of treating cancer comprising administering to a patient in need an effective amount of the antibody or antigen-binding fragment thereof of claim 1.

15. The method of claim 14, wherein the cancer is gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma, or sarcoma.

16. The method of claim 14, wherein the antibody or antigen-binding fragment thereof is administered in combination with another therapeutic agent, wherein the therapeutic agent is an anti-PD1 or anti-PDL1 antibody, paclitaxel or a paclitaxel agent, docetaxel, carboplatin, topotecan, cisplatin, irinotecan, doxorubicin, lenalidomide, or 5-azacytidine.

17.-18. (canceled)

19. An isolated nucleic acid that encodes the anti-CEA antibody or antigen-binding fragment thereof of claim 1.

20. A vector comprising the nucleic acid of claim 19.

21. A host cell comprising the nucleic acid of claim 19.

22. A process for producing an anti-CEA antibody or antigen-binding fragment thereof comprising cultivating the host cell of claim 21 and recovering the antibody or antigen-binding fragment thereof from the culture.


SEQUENCE LISTING (98 sequences)

SEQ ID NO: 1 - protein, 8 aa, synthetic construct
  REGION 1..8: note = BGA113K HCDR1 (IMGT)
GYIFTSYY

SEQ ID NO: 2 - protein, 8 aa, synthetic construct
  REGION 1..8: note = BGA113K HCDR2 (IMGT)
INPQTGKT

SEQ ID NO: 3 - protein, 13 aa, synthetic construct
  REGION 1..13: note = BGA113K HCDR3 (IMGT)
AREYGNYNYPLDY

SEQ ID NO: 4 - protein, 6 aa, synthetic construct
  REGION 1..6: note = BGA113K LCDR1 (IMGT)
ENQYGY

SEQ ID NO: 5 - skipped

SEQ ID NO: 6 - protein, 9 aa, synthetic construct
  REGION 1..9: note = BGA113K LCDR3 (IMGT)/LCDR3 (Kabat)/LCDR3 (Chothia)
QHHLGTPYT

SEQ ID NO: 7 - protein, 5 aa, synthetic construct
  REGION 1..5: note = BGA113K HCDR1 (Kabat)
SYYLH

SEQ ID NO: 8 - protein, 17 aa, synthetic construct
  REGION 1..17: note = BGA113K HCDR2 (Kabat)
YINPQTGKTSYAQKFQG

SEQ ID NO: 9 - protein, 11 aa, synthetic construct
  REGION 1..11: note = BGA113K HCDR3 (Kabat)/HCDR3 (Chothia)
EYGNYNYPLDY

SEQ ID NO: 10 - protein, 11 aa, synthetic construct
  REGION 1..11: note = BGA113K LCDR1 (Kabat)/LCDR1 (Chothia)
RASENQYGYLA

SEQ ID NO: 11 - protein, 7 aa, synthetic construct
  REGION 1..7: note = BGA113K LCDR2 (Kabat)/LCDR2 (Chothia)
NYKNLVE

SEQ ID NO: 12 - protein, 7 aa, synthetic construct
  REGION 1..7: note = BGA113K HCDR1 (Chothia)
GYIFTSY

SEQ ID NO: 13 - protein, 6 aa, synthetic construct
  REGION 1..6: note = BGA113K HCDR2 (Chothia)
NPQTGK

SEQ ID NO: 14 - protein, 120 aa, synthetic construct
  REGION 1..120: note = BGA113K VH
QVQLVQSGAEVKKPGASVKVSCKASGYIFTSYYLHWVRQAPGQGLEWIGYINPQTGKTSYAQKFQGRVTMTRDTSTSTVYMELSSLRSEDTAVYYCAREYGNYNYPLDYWGQGTLVTVSS

SEQ ID NO: 15 - protein, 107 aa, synthetic construct
  REGION 1..107: note = BGA113K VL/BGA113 VL
DIQMTQSPSSLSASVGDRVTITCRASENQYGYLAWYQQKPGKVPKLLIYNYKNLVEGVPSRFSGSGSGTDFTLTISSLQPEDVATYYCQHHLGTPYTFGQGTKVEIK

SEQ ID NO: 16 - DNA, 360 bp, synthetic construct
  misc_feature 1..360: note = BGA113K VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggcaagaccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc

SEQ ID NO: 17 - DNA, 321 bp, synthetic construct
  misc_feature 1..321: note = BGA113K VL DNA/BGA113 VL DNA
  source 1..321: mol_type = other DNA
gatattcagatgacccagagcccgagcagcctgagcgcgagcgtgggcgatcgcgtgaccattacctgccgcgcgagcgaaaaccagtatggctatctggcgtggtatcagcagaaaccgggcaaagtgccgaaactgctgatttataactataaaaacctggtggaaggcgtgccgagccgctttagcggcagcggcagcggcaccgattttaccctgaccattagcagcctgcagccggaagatgtggcgacctattattgccagcatcatctgggcaccccgtatacctttggccagggcaccaaagtggaaattaaa

SEQ ID NO: 18 - protein, 8 aa, synthetic construct
  REGION 1..8: note = BGA190 HCDR1 (IMGT)
GFTFSSFG

SEQ ID NO: 19 - protein, 8 aa, synthetic construct
  REGION 1..8: note = BGA190 HCDR2 (IMGT)
ISIGSDII

SEQ ID NO: 20 - protein, 13 aa, synthetic construct
  REGION 1..13: note = BGA190 HCDR3 (IMGT)
TRRSYRSYWYFDV

SEQ ID NO: 21 - protein, 6 aa, synthetic construct
  REGION 1..6: note = BGA190 LCDR1 (IMGT)
ENIYSY

SEQ ID NO: 22 - skipped

SEQ ID NO: 23 - protein, 9 aa, synthetic construct
  REGION 1..9: note = BGA190 LCDR3 (IMGT)/LCDR3 (Kabat)/LCDR3 (Chothia)
QHHFLSPWM

SEQ ID NO: 24 - protein, 5 aa, synthetic construct
  REGION 1..5: note = BGA190 HCDR1 (Kabat)
SFGMH

SEQ ID NO: 25 - protein, 17 aa, synthetic construct
  REGION 1..17: note = BGA190 HCDR2 (Kabat)
YISIGSDIIYYADTVKG

SEQ ID NO: 26 - protein, 11 aa, synthetic construct
  REGION 1..11: note = BGA190 HCDR3 (Kabat)/HCDR3 (Chothia)
RSYRSYWYFDV

SEQ ID NO: 27 - protein, 11 aa, synthetic construct
  REGION 1..11: note = BGA190 LCDR1 (Kabat)/LCDR1 (Chothia)
RTSENIYSYLA

SEQ ID NO: 28 - protein, 7 aa, synthetic construct
  REGION 1..7: note = BGA190 LCDR2 (Kabat)/LCDR2 (Chothia)
NAKTLAE

SEQ ID NO: 29 - protein, 7 aa, synthetic construct
  REGION 1..7: note = BGA190 HCDR1 (Chothia)
GFTFSSF

SEQ ID NO: 30 - protein, 6 aa, synthetic construct
  REGION 1..6: note = BGA190 HCDR2 (Chothia)
SIGSDI

SEQ ID NO: 31 - protein, 120 aa, synthetic construct
  REGION 1..120: note = BGA190 VH
DVQLVESGGGLVQPGGSRELSCAASGFTFSSFGMHWVRQAPERGLEWVAYISIGSDIIYYADTVKGRFTISRDNPKNTLFLQMTSLRSEDTAVYYCTRRSYRSYWYFDVWGAGTTVTVSS

SEQ ID NO: 32 - protein, 107 aa, synthetic construct
  REGION 1..107: note = BGA190 VL
DIQMTQSPASLSASVGETVTITCRTSENIYSYLAWYQQKQGKSPHLLVYNAKTLAEGVPSRFSGSGSGTQFSLKIISLQPEDFGTYYCQHHFLSPWMFGGGTKLEIK

SEQ ID NO: 33 - DNA, 360 bp, synthetic construct
  misc_feature 1..360: note = BGA190 VH DNA
  source 1..360: mol_type = other DNA
gatgtgcagctggtggagtctgggggaggcttagtgcagcctggagggtcccgggaactctcctgtgcagcctctggattcactttcagtagctttggaatgcactgggttcgtcaggctccagagagggggctggagtgggtcgcatacattagtattggcagtgatatcatctactatgcagacacagtgaagggccgattcaccatctccagagacaatcccaagaacaccctgttcctgcaaatgaccagtctgaggtctgaggacacggccgtgtattactgtacaagaaggtcttataggtcctactggtacttcgatgtctggggcgcagggaccacggtcaccgtctcctca

SEQ ID NO: 34 - DNA, 321 bp, synthetic construct
  misc_feature 1..321: note = BGA190 VL DNA
  source 1..321: mol_type = other DNA
gacatccagatgactcagtctccagcctccctatctgcatctgtgggagaaactgtcaccatcacatgtcgaacaagtgagaatatttacagttatttagcatggtatcagcagaaacagggaaaatctcctcatctcctggtctataatgcaaaaaccttagcagagggtgtgccatcaaggttcagtggcagtggatcaggcacacagttttctctgaagatcatcagcctgcagcctgaagattttgggacttattactgtcagcatcattttcttagtccgtggatgttcggtggaggcaccaagctggaaatcaaa

SEQ ID NO: 35 - protein, 8 aa, synthetic construct
  REGION 1..8: note = BGA288 HCDR1 (IMGT)
GYTFTDYN

SEQ ID NO: 36 - protein, 8 aa, synthetic construct
  REGION 1..8: note = BGA288 HCDR2 (IMGT)
IGPNYGGT

SEQ ID NO: 37 - protein, 12 aa, synthetic construct
  REGION 1..12: note = BGA288 HCDR3 (IMGT)
ARRGSIPRAVDY

SEQ ID NO: 38 - protein, 6 aa, synthetic construct
  REGION 1..6: note = BGA288 LCDR1 (IMGT)
QDIHNY

SEQ ID NO: 39 - skipped

SEQ ID NO: 40 - protein, 9 aa, synthetic construct
  REGION 1..9: note = BGA288 LCDR3 (IMGT)/LCDR3 (Kabat)/LCDR3 (Chothia)
LQYDEFPYT

SEQ ID NO: 41 - protein, 5 aa, synthetic construct
  REGION 1..5: note = BGA288 HCDR1 (Kabat)
DYNID

SEQ ID NO: 42 - protein, 17 aa, synthetic construct
  REGION 1..17: note = BGA288 HCDR2 (Kabat)
DIGPNYGGTIYNQKFKG

SEQ ID NO: 43 - protein, 10 aa, synthetic construct
  REGION 1..10: note = BGA288 HCDR3 (Kabat)/HCDR3 (Chothia)
RGSIPRAVDY

SEQ ID NO: 44 - protein, 11 aa, synthetic construct
  REGION 1..11: note = BGA288 LCDR1 (Kabat)/LCDR1 (Chothia)
KASQDIHNYLS

SEQ ID NO: 45 - protein, 7 aa, synthetic construct
  REGION 1..7: note = BGA288 LCDR2 (Kabat)/LCDR2 (Chothia)
RANRLVD

SEQ ID NO: 46 - protein, 7 aa, synthetic construct
  REGION 1..7: note = BGA288 HCDR1 (Chothia)
GYTFTDY

SEQ ID NO: 47 - protein, 6 aa, synthetic construct
  REGION 1..6: note = BGA288 HCDR2 (Chothia)
GPNYGG

SEQ ID NO: 48 - protein, 119 aa, synthetic construct
  REGION 1..119: note = BGA288 VH
EVLLQQSGPELVKPGASVKIFCKASGYTFTDYNIDWVQQSHGKSLEWIGDIGPNYGGTIYNQKFKGKATLTVAKSSSTAYMELRSLTSEDTAVYYCARRGSIPRAVDYWGQGTSVTVSS

SEQ ID NO: 49 - protein, 107 aa, synthetic construct
  REGION 1..107: note = BGA288 VL
DIKMTQSPSSMYASLGERVTITCKASQDIHNYLSWFQQKPGKSPKTLIYRANRLVDGVPSRFSGSGSGQDYSLTISSLEYEDMGIYYCLQYDEFPYTFGGGTKLEIK

SEQ ID NO: 50 - DNA, 357 bp, synthetic construct
  misc_feature 1..357: note = BGA288 VH DNA
  source 1..357: mol_type = other DNA
gaggtcctgctgcaacagtctggacctgagctggtgaagcctggggcttcagtgaagattttctgtaaggcttctggatacacattcactgactacaacatagactgggtgcagcagagccatggaaagagccttgagtggattggagatattggtcctaattatggtggtactatctacaaccagaagttcaagggcaaggccacattgactgtagccaagtcctccagcacagcctacatggagctccgcagcctgacatctgaggacactgcagtctattactgtgcaagacgcggtagcatcccgagggctgtggactactggggtcaaggaacctcagtcaccgtctcctca

SEQ ID NO: 51 - DNA, 321 bp, synthetic construct
  misc_feature 1..321: note = BGA288 VL DNA
  source 1..321: mol_type = other DNA
gacatcaagatgacccagtctccatcttccatgtatgcatctctaggagagagagtcactatcacttgcaaggcgagtcaggacattcataactatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggggggaccaagctggaaataaaa

SEQ ID NO: 52 - protein, 702 aa, Homo sapiens
  REGION 1..702: note = full-length human CEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 53 - protein, 705 aa, Macaca mulatta
  REGION 1..705: note = Macaca CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARATVGIIIGMLVGVALM

SEQ ID NO: 54 - protein, 344 aa, Homo sapiens
  REGION 1..344: note = full-length human CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 55 - protein, 693 aa, synthetic construct
  REGION 1..693: note = 1-687 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 56 - protein, 696 aa, synthetic construct
  REGION 1..696: note = 1-690 of monkey CEA
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLTCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNSGAYACFVSNLATGRNNSIVKNISVSSGDSAPGSSGLSARAHHHHHH

SEQ ID NO: 57 - protein, 326 aa, synthetic construct
  REGION 1..326: note = 1-320 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGHHHHHH

SEQ ID NO: 58 - protein, 115 aa, synthetic construct
  REGION 1..115: note = 1-78 of huCEA
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQN

SEQ ID NO: 59 - protein, 290 aa, synthetic construct
  REGION 1..290: note = 398-687 of huCEA
ELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 60 - protein, 411 aa, synthetic construct
  REGION 1..411: note = CEA-v
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 61 - protein, 243 aa, synthetic construct
  REGION 1..243: note = 1-273 of CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTIS

SEQ ID NO: 62 - protein, 92 aa, synthetic construct
  REGION 1..92: note = 596-687 of huCEA
PIISPPDSSYLSGANLNLSCHSASNPSPQYSWRINGIPQQHTQVLFIAKITPNNNGTYACFVSNLATGRNNSIVKSITVSASGTSPGLSAGA

SEQ ID NO: 63 - protein, 341 aa, synthetic construct
  REGION 1..341: note = CHIM
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPIISPPDSSYLSGANLNLSCHSASNPSPQYSWRINGIPQQHTQVLFIAKITPNNNGTYACFVSNLATGRNNSIVKSITVSASGTSPGLSAGAHHHHHH

SEQ ID NO: 64 - protein, 464 aa, synthetic construct
  REGION 1..464: note = CEACAM1
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGSSGPLQ

SEQ ID NO: 65 - protein, 252 aa, synthetic construct
  REGION 1..252: note = CEACAM3
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 66 - protein, 265 aa, synthetic construct
  REGION 1..265: note = CEACAM7
MGSPSACPYRVCIPWQGLLLTASLLTFWNLPNSAQTNIDVVPFNVAEGKEVLLVVHNESQNLYGYNWYKGERVHANYRIIGYVKNISQENAPGPAHNGRETIYPNGTLLIQNVTHNDAGIYTLHVIKENLVNEEVTRQFYVFSEPPKPSITSNNFNPVENKDIVVLTCQPETQNTTYLWWVNNQSLLVSPRLLLSTDNRTLVLLSATKNDIGPYECEIQNPVGASRSDPVTLNVRYESVQASSPDLSAGTAVSIMIGVLAGMALI

SEQ ID NO: 67 - protein, 349 aa, synthetic construct
  REGION 1..349: note = CEACAM8
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 68 - DNA, 2109 bp, synthetic construct
  misc_feature 1..2109: note = full-length human CEA DNA
  source 1..2109: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 69 - DNA, 2118 bp, synthetic construct
  misc_feature 1..2118: note = monkey CEA DNA
  source 1..2118: mol_type = other DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctactgtcggcatcataattggaatgctggttggggttgctctgatgtag

SEQ ID NO: 70 - DNA, 1035 bp, synthetic construct
  misc_feature 1..1035: note = full-length human CEACAM6 DNA
  source 1..1035: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatag

SEQ ID NO: 71 - DNA, 2079 bp, synthetic construct
  misc_feature 1..2079: note = 1-687 of huCEA DNA
  source 1..2079: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 72 - DNA, 2088 bp, synthetic construct
  misc_feature 1..2088: note = 1-690 of monkey CEA DNA
  source 1..2088: mol_type = other DNA
atggggtctccctcagcccctcttcacagatggtgcatcccctggcagacgctcctgctcacagcctcacttctaaccttctggaacccgcccaccactgcccagctcactattgaatccaggccgttcaatgttgcagaggggaaggaggttcttctacttgcccacaatgtgtcccagaatctttttggctacatttggtacaagggagaaagagtggatgccagccgtcgaattggatcatgtgtaataagaactcaacaaattaccccagggcccgcacacagcggtcgagagacaatagacttcaatgcatccctgctgatccacaatgtcacccagagtgacacaggatcctacaccatacaagtcataaaggaagatcttgtgaatgaagaagcaactggccagttccgggtatacccggagctgcccaagccctacatctccagcaacaactccaaccccgtggaggacaaggatgctgtggccttaacctgtgaacctgagactcaggacacaacctacctgtggtgggtaaacaatcagagcctcccggtcagtcccaggctggagctgtccagtgacaacaggaccctcactgtattcaatattccaagaaatgacacaacatcctacaaatgtgaaacccagaacccagtgagtgtcagacgcagcgacccagtcaccctgaacgtcctctatggcccggatgcgcccaccatttcccctctaaacacaccttacagagcaggggaaaatctgaacctcacctgccacgcagcctctaacccaactgcacagtacttttggtttgtcaatgggacgttccagcaatccacacaagagctctttatacccaacatcaccgtgaataatagcggatcctatatgtgccaagcccataactcagccactggcctcaataggaccacagtcacggcgatcacagtctacgcggagctgcccaagccctacatcaccagcaacaactccaaccccatagaggacaaggatgctgtgaccttaacctgtgaacctgagactcaggacacaacctacctgtggtgggtaaacaatcagagcctctcggtcagttccaggctggagctgtccaatgacaacaggaccctcactgtattcaatattccaagaaacgacacaacgttctacgaatgtgagacccagaacccagtgagtgtcagacgcagcgacccagtcaccctgaatgtcctctatggcccggatgcgcccaccatttcccctctaaacacaccttacagagcaggggaaaatctgaacctctcctgccacgcagcctctaacccagctgcacagtactcttggtttgtcaatgggacgttccagcaatccacacaagagctctttatacccaacatcaccgtgaataatagcggatcctatatgtgccaagcccataactcagccactggcctcaataggaccacagtcacggcgatcacagtctatgtggagctgcccaagccctacatctccagcaacaactccaaccccatagaggacaaggatgctgtgaccttaacctgtgaacctgtggctgagaacacaacctacctgtggtgggtaaacaatcagagcctctcggtcagtcccaggctgcagctctccaatggcaacaggatcctcactctactcagtgtcacacggaatgacacaggaccctatgaatgtggaatccagaactcagagagtgcaaaacgcagtgacccagtcaccctgaatgtcacctatggcccagacacccccatcatatcccccccagacttgtcttaccgttcgggagcaaacctcaacctctcctgccactcggactctaacccatccccgcagtattcttggcttatcaatgggacactgcggcaacacacacaagttctctttatctccaaaatcacatcaaacaatagcggggcctatgcctgttttgtctctaacttggctaccggtcgcaataactccatagtcaagaacatctcagtctcctctggcgattcagcacctggaagttctggtctctcagctagggctcatcaccatcaccatcac

SEQ ID NO: 73 - DNA, 978 bp, synthetic construct
  misc_feature 1..978: note = 1-320 of CEACAM6 DNA
  source 1..978: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggacatcaccatcaccatcac

SEQ ID NO: 74 - DNA, 345 bp, synthetic construct
  misc_feature 1..345: note = 1-78 of huCEA DNA
  source 1..345: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaat

SEQ ID NO: 75 - DNA, 870 bp, synthetic construct
  misc_feature 1..870: note = 398-687 of huCEA DNA
  source 1..870: mol_type = other DNA
gaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 76 - DNA, 1233 bp, synthetic construct
  misc_feature 1..1233: note = CEA-v DNA
  source 1..1233: mol_type = other DNA
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 77 - DNA, 729 bp, synthetic construct
  misc_feature 1..729: note = 1-273 of CEACAM6 DNA
  source 1..729: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcc

SEQ ID NO: 78 - DNA, 276 bp, synthetic construct
  misc_feature 1..276: note = 596-687 of huCEA DNA
  source 1..276: mol_type = other DNA
cccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcc

SEQ ID NO: 79 - DNA, 1023 bp, synthetic construct
  misc_feature 1..1023: note = CHIM DNA
  source 1..1023: mol_type = other DNA
atgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttcccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggcccatcaccatcaccatcac

SEQ ID NO: 80 - protein, 8 aa, synthetic construct
  REGION 1..8: note = BGA13 HCDR1 (IMGT)
GYIFTSYW

SEQ ID NO: 81 - protein, 8 aa, synthetic construct
  REGION 1..8: note = BGA13 HCDR2 (IMGT)
INPNTGYT

SEQ ID NO: 82 - protein, 6 aa, synthetic construct
  REGION 1..6: note = BGA13 LCDR1 (IMGT)
ENIYGY

SEQ ID NO: 83 - skipped

SEQ ID NO: 84 - protein, 9 aa, synthetic construct
  REGION 1..9: note = BGA13 LCDR3 (IMGT)
QHHYGTPYT

SEQ ID NO: 85 - protein, 120 aa, synthetic construct
  REGION 1..120: note = BGA13 VH
QVQLQQSGAELAKSGASVKMSCKASGYIFTSYWLHWVKQRPGQGLEWIGYINPNTGYTNYSQKFKDKATLTADKSSSTAYMQLSSLTSEDSAVYYCAREYGNYNYPLDYWGQGTSVTVSS

SEQ ID NO: 86 - protein, 107 aa, synthetic construct
  REGION 1..107: note = BGA13 VL
DIQMTQSPASLSASVGETVTITCRASENIYGYLAWYQQKQGKSPQLLVFNAKNLVEGVPSRFSGSGSGTQFSLKINSLQPEDFGSYYCQHHYGTPYTFGGGTKLEIK

SEQ ID NO: 87 - protein, 330 aa, Homo sapiens [sequence omitted]

SEQ ID NO: 88 - protein, 107 aa, synthetic construct [sequence omitted]

SEQ ID NO: 89 - protein, 450 aa, synthetic construct
  REGION 1..450: note = BGA131 heavy chain
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  REGION 1..214: note = BGA131 light chain
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 120 aa, synthetic construct
  REGION 1..120: note = BGA131 VH
QVQLVQSGAEVKKPGASVKVSCKASGYIFTSYWLHWVRQAPGQGLEWIGYINPNTGYTNYSQKFQGRVTMTRDTSTSTVYMELSSLRSEDTAVYYCAREYGNYNYPLDYWGQGTLVTVSS

SEQ ID NO: 92 - protein, 107 aa, synthetic construct
  REGION 1..107: note = BGA131 VL
DIQMTQSPSSLSASVGDRVTITCRASENIYGYLAWYQQKPGKVPKLLIYNAKNLVEGVPSRFSGSGSGTDFTLTISSLQPEDVATYYCQHHYGTPYTFGQGTKVEIK

SEQ ID NO: 93 - protein, 107 aa, synthetic construct; identical to SEQ ID NO: 92
  REGION 1..107: note = BGA13-1 VL

SEQ ID NO: 94 - protein, 120 aa, synthetic construct
  REGION 1..120: note = BGA13-1F VH
QVQLVQSGAEVKKPGASVKVSCKASGYIFTSYWLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 95 - protein, 107 aa, synthetic construct
  REGION 1..107: note = BGA13-1F-ph-L VL
DIQMTQSPSSLSASVGDRVTITCRASENQYGYLAWYQQKPGKVPKLLIYNFKNLVEGVPSRFSGSGSGTDFTLTISSLQPEDVATYYCQHHLGTPYTFGQGTKVEIK

SEQ ID NO: 96 - protein, 107 aa, synthetic construct
  REGION 1..107: note = BGA13-1F-ph-M VL
DIQMTQSPSSLSASVGDRVTITCRASENQYGYLAWYQQKPGKVPKLLIYNTKNLVEGVPSRFSGSGSGTDFTLTISSLQPEDVATYYCQHHLGTPYTFGQGTKVEIK

SEQ ID NO: 97 - protein, 120 aa, synthetic construct
  REGION 1..120: note = BGA113 VH
QVQLVQSGAEVKKPGASVKVSCKASGYIFTSYYLHWVRQAPGQGLEWIGYINPQTGYTSYAQKFQGRVTMTRDTSTSTVYMELSSLRSEDTAVYYCAREYGNYNYPLDYWGQGTLVTVSS

SEQ ID NO: 98 - DNA, 360 bp, synthetic construct
  misc_feature 1..360: note = BGA113 VH DNA
  source 1..360: mol_type = other DNA
caggtgcagctggtgcagagcggcgcggaagtgaaaaaaccgggcgcgagcgtgaaagtgagctgcaaagcgagcggctatatttttaccagctattacctgcattgggtgcgccaggcgccgggccagggcctggaatggattggctatattaacccgcagaccggctataccagctatgcccagaaatttcagggccgcgtgaccatgacccgcgataccagcaccagcaccgtgtatatggaactgagcagcctgcgcagcgaagataccgcggtgtattattgcgcgcgcgaatatggcaactataactatccgctggattattggggccagggcaccctggtgaccgtgagcagc